(19) United States
(12) Patent Application Publication
(10) Pub. No.: US 2021/0030734 A1
(43) Pub. Date: Feb. 4, 2021

(54) METHOD OF TREATING AMYOTROPHIC LATERAL SCLEROSIS WITH PRIDOPIDINE

(71) Applicant: PRILENIA NEUROTHERAPEUTICS LTD., Herzliya (IL)

(72) Inventors: Michal GEVA, Even-Yehuda (IL); Michael Hayden, Herzliya (IL)

(73) Assignee: PRILENIA NEUROTHERAPEUTICS LTD., Herzliya (IL)

(21) Appl. No.: 17/076,069

(22) Filed: Oct. 21, 2020

Related U.S. Application Data
(63) Continuation-in-part of application No. 16/789,564, filed on Feb. 13, 2020.
Application No. 16/789,564 is a continuation-in-part of application No. PCT/US2018/046481, filed on Aug. 13, 2018.
(60) Provisional application No. 62/545,315, filed on Aug. 14, 2017.

(51) Int. Cl. A61K 31/451; A61P 25/28; A61K 9/00

(52) U.S. Cl. CPC A61K 31/451; A61K 45/06; A61K 9/0053; A61P 25/28

(57) ABSTRACT

Provided herein is a method for treating a human subject afflicted with ALS by administering to the subject a therapeutically effective amount of pridopidine.

### CROSS REFERENCE TO RELATED APPLICATIONS

This Application is a Continuation-in-Part Application from U.S. application Ser. No. 16/789,564 filed Feb. 13, 2020, which is a Continuation-in-Part Application from International Patent Application No. PCT/US2018/046481 filed Aug. 13, 2018, which claims the benefit of U.S. Provisional Application No. 62/545,315, filed Aug. 14, 2017 the entire content of which are hereby incorporated by reference herein.

### BACKGROUND OF THE INVENTION

### Amyotrophic Lateral Sclerosis

Amyotrophic lateral sclerosis (ALS) is a devastating degenerative disease characterized by progressive loss of motor neurons in the motor cortex, brainstem, and spinal cord (Peters 2015). This rapidly progressing fatal disease leads to weakness of limb, respiratory, and bulbar muscles. Patients progressively lose control of voluntary muscles, leading to loss of limb function and the ability to chew, swallow, speak and eventually breathe.

ALS is a rare condition, having a mean incidence rate of 2.8/100,000 in Europe and 1.8/100,000 in North America, and a mean prevalence rate of 5.40/100,000 in Europe and 3.40/100,000 in North America (Bozzoni 2016).

About 10% of ALS cases are classified as familial (FALS), whereas the remaining 90% are classified as sporadic (sALS) and occur randomly (Riva 2016). Over 60% of patients die within 3 years of presentation, usually from respiratory failure and about 10% survive for more than 10 years (Zou 2016). There is currently no known disease-modifying therapy for ALS, although riluzole slows the rate of progression of the disease and prolongs survival by 2 or 3 months and edaravone slows physical decline (Jaiswal 2018).

Clinical manifestations of ALS include muscle weakness and hypotrophy, fasciculations and cramps, spastic hypertonus, and hyperreflexia are the main clinical manifestations. Some patients also display dysarthria, dysphagia and respiratory weakness. Non-motor symptoms include behavioral disturbances, dysexecutive impairment, and frontotemporal dementia.

The neuropathological features of ALS include muscle atrophy, loss of anterior horn cells, and sclerosis of the spinal cord lateral columns (Martel 2016). Gliosis, defined as activation of astrocytes and microglia, is also a hallmark of ALS.

### Pridopidine

Pridopidine (formerly ACR16, Huntexil®) chemical name of pridopidine is 4-(3-(Methylsulfonyl)phenyl)-1-propylpiperidine, and its Chemical Registry Number is CAS 346688-38-8 (CSID:7971505, 2016). The Chemical Registry number of pridopidine hydrochloride is 882737-42-0 (CSID:25948790 2016). Processes of synthesis of pridopidine and a pharmaceutically acceptable salt thereof are disclosed in U.S. Pat. No. 7,923,459 and PCT Application Publication No. WO 2017/015609. U.S. Pat. No. RE46,117 discloses pridopidine for the treatment of a variety of diseases and disorders.

Pridopidine is a highly selective S1R ligand which has ˜30-fold higher affinity towards the S1R vs D3Rs, and ˜500-fold higher affinity vs D2Rs. Selective binding of pridopidine for the S1R with no dopamine D2/D3R binding was confirmed using positron emission tomography (PET) imaging in rats (Sahlholm, 2015), and in humans at low doses (45 mg BID) (TV7820-IMG-10082). The neuroprotective properties of pridopidine are mediated by its activation of the S1R, as its silencing by genetic or pharmacological methods abolishes the protective effects of pridopidine (Eddings 2019, Ryskamp 2018, Ionescu 2019).

The S1R is a highly conserved transmembrane protein located in the endoplasmic reticulum (ER) and specifically enriched in the subregions contacting mitochondria (Mitochondria-Associated Membranes, MAM). The S1R is highly enriched in the CNS and specifically within the Basal Ganglia, cortex, spinal cord, and brainstem. The S1R is implicated in cellular differentiation, neuroplasticity, neuroprotection and cognitive function in the brain.

Mice with a deletion of the S1R, gene (S1R−/−) display impairments in motor function similar to ALS model mice. Motor neurons (MNs) from these mice also exhibit reduced axonal length and survival (Bernard-Marissal, 2015). In the ALS SOD1^{G93A} mouse model that also lacks S1R expression (SOD1^{G93A}/S1R−/−), disease progression is accelerated, resulting in earlier symptom onset and decreased survival (Mavlyutov, 2013). Pharmacological activation of the S1R increases survival and improves motor function in ALS mice (Mancuso, 2012).

Post-mortem analysis of spinal cord tissue from ALS patients reveals that S1R levels are reduced and abnormally localized (Mavlyutov 2013). In the remaining MNs, local elevation in S1R levels is detected in the enlarged C-terminals, suggesting a compensatory process to preserve the function of S1R at these sites (Prause, 2013).

Furthermore, it is surprising and unexpected that loss of function mutations causing complete absence of the S1R protein result in juvenile ALS (severe form) and missense mutations resulting in partial loss of function, are causative of adult onset ALS. This suggests a dose dependent correlation between S1R function and age of onset (Al-Saif, 2011; Izumi, 2018; Watanabe, 2016).

Recently, transcriptomic analysis of rat striatum showed that pridopidine treatment activates expression of the BDNF, dopamine receptor 1 (D1R), glucocorticoid receptor (GR), and the serine-threonine kinase protein kinase B (Akt)/phosphoinositi de 3-kinase (P13K) pathways. Pridopidine was shown to enhance secretion of the neuroprotective brain-derived neurotrophic factor (BDNF) in a neuroblastoma cell line, in a S1R-dependent manner (Geva 2016) and to rescue spine impairment and aberrant calcium signaling by activation of the S1R (Ryskamp 2017).

In ALS SOD1^{G93A} motor neurons (MNs), pridopidine increases survival, rescues impaired BDNF and mitochondrial axonal transport, and restores neuromuscular junction (NMJ) morphology and synaptic activity. The protective effects of pridopidine are abolished after genetic deletion of S1Rs. In vivo, pridopidine treatment of SOD1^{693A} mice reduces toxic protein aggregates and ameliorates muscle fiber wasting (examples in this application, Ionescu, 2019).

There remains an unmet need to provide effective treatments for ALS, in particular sporadic ALS.

### SUMMARY OF THE INVENTION

The present invention is based, at least in part, on the surprising experimental discovery that pridopidine treatment improves axonal transport deficits, enhances ERK activation and restores neuromuscular junction (NMJ) activity in SOD1 impaired muscle cell co-cultures, reduces mutant SOD1 aggregates in the spinal cord, and attenuates NW disruption and subsequent muscle wasting in SOD1 impaired mice.

The invention provides a method for treating a subject afflicted with amyotrophic lateral sclerosis (ALS), comprising periodically administering to the subject an amount of pridopidine effective to treat the human subject.

The invention also provides for a method for maintaining, improving, or lessening the decline of ALS patient's functionality, respiratory function, muscle strength, bulbar function, speech or any combination thereof in a subject afflicted with amyotrophic lateral sclerosis (ALS), comprising administering to the subject a therapeutically acceptable amount of pridopidine. The invention also provides for pridopidine for use in treating a human subject afflicted with ALS.

The invention also provides a method for treatment of ALS comprising periodically administering to a subject in need thereof an amount of pridopidine effective to treat the ALS.

In some embodiments the subject is afflicted with sporadic ALS.

The invention also provides for a pharmaceutical composition for treating a human subject afflicted with ALS comprising an effective amount of pridopidine.

The invention also provides for the use of pridopidine in the manufacture of a medicament for the treatment of ALS.

Further provided is a package comprising pridopidine and optionally instructions for using the pridopidine to treat ALS.

In one embodiment, pridopidine is administered as a monotherapy to a subject afflicted with ALS.

In other embodiments, a subject afflicted with ALS is treated with pridopidine and with one or more additional agents.

Further provided is a method of treating a subject afflicted with ALS comprising periodically administering to the subject an amount of riluzole, and an amount of pridopidine.

Further provided is a method of treating a subject afflicted with ALS comprising periodically administering to the subject an amount of edaravone, and an amount of pridopidine

Further provided is a method of treating a subject afflicted with ALS comprising periodically administering to the subject an amount of dextromethorphan/quinidine and an amount of pridopidine.

Further provided is a method of treating a subject afflicted with ALS comprising periodically administering to the subject an amount of Zilucoplan and an amount of pridopidine.

Further provided is a method of treating a subject afflicted with ALS comprising periodically administering to the subject an amount of Verdiperstat and an amount of pridopidine.

Further provided is a method of treating a subject afflicted with ALS comprising periodically administering to the subject an amount of CNM-Au8 nanocrystalline gold and an amount of pridopidine.

Further provided is a method of treating a subject afflicted with ALS comprising periodically administering to the subject an amount of IC14 nanocrystalline gold and an amount of pridopidine.

Further provided is a method of treating a subject afflicted with ALS comprising administering to the subject an amount of sodium phenylbutyrate (PB) or tauroursodeoxvcbolic acid (TUDGA) and an amount of pridopidine.

Further provided is a method of treating a subject afflicted with ALS comprising administering to the subject an amount of combination of sodium phenylbutyrate (PB) and tauroursodeoxycholic acid (i.e. AMX0035) and an amount of pridopidine.

Further provided is a package comprising:
a) a first pharmaceutical composition comprising an amount of an agent which is riluzole, edaravone, combination of dextromethorphan/quinidine, sodium phenylbutyrate (PB), tauroursodeoxycholic acid or a combination of sodium phenyibutyrate (PB)/tauroursodeoxycholic acid (i.e. AMX0035), Zilucoplan, Verdiperstat, CNM-Au8 nanocrystalline gold, IC14; and
b) a second pharmaceutical composition comprising and amount of pridopidine and a pharmaceutically acceptable carrier.

In a further embodiment, the package also comprises:
c) instructions for use for the first and the second pharmaceutical compositions together to treat a subject afflicted with ALS.

In some embodiments, the first pharmaceutical composition is riluzole, edaravone, a combination of dextromethorphan/quinidine, laquinimod, sodium phenylbutyrate (PB), tauroursodeoxycholic acid, a combination of sodium phenylbutyrate (PB)/tauroursodeoxycholic acid (i.e.AMX0035), Zilucoplan, Verdiperstat, CNM-Au8 nanocrystalline gold or IC14.

Further provided is pridopidine for use as an add-on therapy of or in combination with riluzole in treating a subject afflicted with ALS. Further provided is pridopidine for use as an add-on therapy of or in combination with edaravone in treating a subject afflicted with ALS. Further provided is pridopidine for use as an add-on therapy of or in combination with dextromethorphaniquinidine in treating a subject afflicted with ALS. Further provided is pridopidine for use as an add-on therapy of or in combination with sodium phenylbutyrate (PB) in treating a subject afflicted with ALS. Further provided is pridopidine for use as an add-on therapy of or in combination with tauroursodeoxychol c acid in treating a subject afflicted with ALS. Further provided is pridopidine for use as an add-on therapy of or in combination with a combination of sodium phenylbutyrate (PB)/tauroursodeoxycholic acid (i.e. AMX0035) in treating a subject afflicted with ALS. Further provided is pridopidine for use as an add-on therapy of or in combination with a combination of Zilucoplan. Further provided is pridopidine for use as an add-on therapy of or in combination with a combination of Verdiperstat. Further provided is pridopidine for use as an add-on therapy of or in combination with a combination of CNM-Au8 nanocrystalline gold. Further provided is pridopidine for use as an add-on therapy of or in combination with a combination of IC14.

The subject invention also provides a pharmaceutical composition comprising an amount of riluzole, edaravone combination of dextromethorphan/quinidine, sodium phenylbutyrate (PB), tauroursodeoxycholic acid, Zilucoplan, Verdiperstat, CNM-Au8 nanocrystalline gold, IC14, or a combination of sodium phenylbutyrate (PB)/tauroursodeoxycholic acid (i.e. AMX0035), and an amount of pridopidine, and at least one pharmaceutical acceptable carrier.

The subject invention also provides the use of:
a) an amount of riluzole, edaravone, combination of dextromethorphanlquinidine, sodium phenylbutyrate (PB), tauroursodeoxycholic acid, Zilucoplan, Verdiperstat, CNM-Au8 nanocrystalline gold, IC14, or combination of sodium phenyibutyrate (PB)/tauroursodeoxycholic acid (i.e. AMX0035); and
b) an amount of pridopidine
   in the preparation of a combination for treating a subject afflicted with ALS wherein the amount of riluzole, edaravone, combination of dextromethorphan/quinidine, sodium phenylbutyrate (PB), tauroursodeoxycholic acid, combination of sodium phenyibutyrate (PB)/tauroursodeoxycholic acid (i.e. AMX0035), Zilucoplan, Verdiperstat, CNM-Au8 nanocrystalline gold or IC14, and the amount of pridopidine are administered simultaneously or contemporaneously.

The subject invention also provides a pharmaceutical composition comprising an amount of riluzole, edaravone, dextromethorphan/quinidine or sodium phenylbutyrate (PB), tauroursodeoxycholic acid, Zilucoplan, Verdiperstat, CNM-Au8 nanocrystalline gold, IC14, or sodium phemdbutwate (PB)/tauroursodcoxycholic acid (i.e. AMX0035) for use in treating a subject afflicted with a movement disorder, in combination with an amount of pridopidine, by periodically administering to the subject the pharmaceutical composition and the amount of pridopidine.

The subject invention also provides a pharmaceutical composition comprising an amount of pridopidine for use treating a subject afflicted with a movement disorder, in combination with an amount of riluzole, edaravone, dextromethorphan/quinidine, sodium phenylbutyrate (PB), tauroursodeoxycholic acid, Zilucoplan, Verdiperstat, CNM-Au8 nanocrystalline gold, IC14, or sodium phenylbutyrate (PB)/tauroursodcoxyclioic acid (i.e. AMX0035), by periodically administering to the subject the pharmaceutical composition and the amount of riluzole, edaravone, dextromethorphan/quinidine, sodium phenylbutyrate (PB), tauroursodeoxycholic acid, Zilucoplan, Verdiperstat, CNM-Au8 nanocrystalline gold, IC14, or sodium plienylbutyrate (PB)/tauroursodeoxycholic acid (i.e. AMX0035), respectively.

The subject invention also provides riluzole and pridopidine for the treatment of a subject afflicted with a movement disorder, wherein the riluzole and the pridopidine are administered simultaneously, or contemporaneously.

The subject invention also provides a product containing an amount of riluzole and an amount of pridopidine for simultaneous or contemporaneous use in treating a subject afflicted with ALS.

In another aspect, the invention provides a combination of pridopidine, and riluzole, edaravone, combination of dextromethorphan/quinidine, sodium phenylbutyrate (PB), tauroursodeoxycholic acid, Zilucoplan, Verdiperstat, CNM-Au8 nanocrystalline gold, IC14, or combination of sodium phenyihutyrate (PB)/tauroursodeoxycholic acid (i.e. AMX0035), for use as a medicament for the treatment, prevention or alleviation of ALS.

In another aspect the invention provides a combination of pridopidine, and riluzole, edaravone, laquinimod, combination of dextromethorphan/quinidine, sodium phenylbutyrate (PB), tauroursodeoxycholic acid, Zilucoplan, Verdiperstat, CNM-Au8 nanocrystalline gold, IC14, or combination of sodium phenylbutyrate (PB)/tauroursodeoxycholic acid (i.e. AMX0035), for use as a medicament.

In another aspect the invention provides a pharmaceutical composition comprising a therapeutically effective amount of pridopidine, and a therapeutically effective amount of riluzole, edaravone, laquinimod, combination of dextromethorphan/quinidine, sodium phenyibutyrate (PB), tauroursodeoxycholic acid, Zilucoplan, Verdiperstat, CNM-Au8 nanocrystalline gold, IC14, or combination of sodium phenylbutyrate (PB)/tauroursodeoxycholic add (i.e. AMX0035), together with one or more adjuvants, excipients, carriers and/or diluents.

In another aspect the invention provides a method of treating of ALS in a living animal body, including a human, which method comprises the step of administering to such a living animal body in need thereof, a therapeutically effective amount of pridopidine; in a combination therapy with riluzole, edaravone, laquinimod, combination of dextromethorphan/quinidine, sodium phenyibtorate (PB), tauroursodeoxycholic acid, Zilucoplan, Verdiperstat, CNM-Au8 nanocrystalline gold, IC14 or combination of sodium phenyibutvrate (PB)/tauroursodeoxycholic acid (i.e. AMX0035).

In another aspect the invention provides a kit of parts comprising at least two separate unit dosage forms (A) and (B), wherein (A) comprises pridopidine; and (B) comprises riluzole, edaravone, combination of dextromethorphan/quinidine, sodium phenylbutyrate (PB), tauroursodeoxycholic acid, Zilucoplan, Verdiperstat, CNM-Au8 nanocrystalline gold, IC14 or combination of sodium phenylbutyrate (PB)/tauroutsodeoxycholic acid (i.e. AMX0035); and optionally (C) instructions for the simultaneous or contemporaneous administration of the pridopidine of (A) and the riluzole, edaravone, combination of dextromethorphan/quinidine, sodium phenyibutyrate (PB), tauroursodeoxycholic acid, Zilucoplan, Verdiperstat, CNM-Au8 nanocrystalline gold, IC14, or combination of sodium phenylbutyrate (PB)/tauroursodeoxycholic acid (i.e. AMX0035) of (B), to a patient in need thereof.

In another aspect the invention provides a method of treating a subject afflicted with ALS comprising administering to the subject a combination of a therapeutically effective amount of pridopidine, and a therapeutically effective amount of riluzole, edaravone, laquinimod, combination of dextromethorphanlquinidine, sodium phenylbutyrate (PB), tauroursodeonicholic acid, Zilucoplan, Verdiperstat, CNM-Au8 nanocrystalline gold, IC14, or combination of sodium phenylbutyrate (PB/tauroursodeoxycholic acid (i.e. AMX0035), wherein the amounts when taken together are effective to treat the human patient.

### BRIEF DESCRIPTION OF THE FIGURES

FIGS. 1A-1C. Axonal transport assay. FIG. 1A. Experimental workflow for the axonal transport assay. FIG. 1B, Schematic illustration of the experimental system for axonal transport tracking in motor neurons (MNs). FIG. 1C. Time lapse images of Qdot-BDNF (marked with arrow) axonal transport.

FIGS. 2A-2B. Graphs showing effect of pridopidine on instantaneous velocity values and particle stop count of Qdot-BDNF along the axon (WT=wild type) FIG. 2A. Pridopidine's effect on instantaneous velocity values (μm/sec) for Qdot-BDNF particles in WT, SOD1G93A or Sigma-1 receptor knock out (S1R−/−) MNs. FIG. 29. Pridopidine's effect on particle stop count (number of counted stops of Qdot-BDNF per second). Data are shown as the mean±SEM. *p value <0.05; **p value <0.01; ***p value <0.001 (n=6 independent experiments; the sample size for each experiment is indicated on bars; Student's t test)

FIG. 3. Schematic illustration of the experimental procedure for neuromuscular coculture assays measuring muscle innervation and Neuro Muscular Junction function (NMJ). Spinal cord explant is cultured in the proximal compartment and primary myocytes are cultured in the distal compartment.

FIG. 4. Graph showing pridopidine's (1 μM) effect on axonal growth for WI and SOD1G93A (SOD1) ALS, which measures the number of grooves with axons crossing into the muscle compartment.

FIGS. 5A and 5B. Results of Micro Fluidic Compartment (MFC) co-culture assays FIG. 5A. Upper panel: Phase image of a myocyte in the distal compartment connected by axons (arrowheads). Lower panel: High magnification images of myocyte: MN contact points. Inset: rendering of colocalization. FIG. 5B. Muscle contraction traces as extracted from intensity over time measurements of muscle contraction.

FIG. 6. Graph showing pridopidine's effect on axonal innervation rate in WT and SOD1G93A (SOD1) ALS myocytes.

FIGS. 7A-7B. Graphs showing level of contracting myocytes in motor neuron (MN)-myocyte co-culture—neuromuscular junction (NMJ). FIG. 7A. The effect of pridopidine on percent of contracting myoblasts in co-cultures of ventral spinal cord (WT or SOD1G93A) sections and primary myocytes (SOD1G93A or WT controls). FIG. 7B. Pridopidine's effect on contracting myocytes in muscle tissue from WT or SOD1G93A and motor neurons from WI or S1R−/− mice. Data are shown as mean±SEM. *p value <0.05; **p value <0.01, ***p value <0.001, ****p value <0.0001. (n=number of microfluidic chambers from 3 or more independent experiments; Student's t test)

FIGS. 8A-8B. Effect of pridopidine on ERK levels. FIG. 8A. Pridopidine's effect on total ERK (tERK) and phosphorylated ERK (pERK) levels shown in Western blots from WT, SOD1G93A, and S1R−/− MNs culture extracts. FIG. 8B. Quantification of pridopidine's effect of ERK activation as measured by tERK/pERK. Data are shown as the mean pERK/ERK±SEM. *p value <0.05, ˜p value <0.1 (n=3 independent experiments; Student's t test.)

FIGS. 9A-9C. Effect of pridopidine on mutant SOD1 aggregates. FIG. 9A. Visualization and quantification of fluorescently labeled spinal cords with NC500 to label mutant SOD1 aggregates in WT and SOD1G93A (ALS) spinal cords, from mice treated or not with pridopidine. FIG. 9B. Quantitative analysis of the number of SOD1 aggregates per area identified in the gray matter. FIG. 9C. Quantitative analysis of the number of SOD1 aggregates per area identified in the white matter. Data are shown as the mean±SEM. *p value <0.05; **p value <0.01 (n=4 mice in each group; one-way ANOVA followed by Fisher's LSD post hoc tests.)

FIGS. 10A-10B. Effect of pridopidine on muscle tissue. FIG. 10A. Representative images of H&E-stained cross sections from Gastrocnemius muscle of WT or SOD1G93A mice treated or is not with Pridopidine. FIG. 10B. Assessment of pridopidine's effect on muscle fiber wasting: quantitative analysis of pridopidine's effect on muscle fiber diameter. Data are shown as mean±SEM (n=number of NMJs). *p value <0.05; **p value <0.01; ***p value <0.001 (n=5 mice in each group; Student's t test)

FIGS. 11A-11B. Effect of pridopidine on NMJ. FIG. 11A. Immuno-staining of Pridopidine's effect NMJ preservation in vivo. Top panel: Neurofilament heavy (NFH) and synapsin I (SynP) antibodies mark the neuronal, presynaptic side of the NMJ. Middle panel: Bungarotoxin (BTX) specifically marks the acetyl choline receptor on the muscle side of the NMJ. Bottom panel: An overlay of the neuronal and muscular markers shows an overlap at NMJ sites. FIG. 11B. Quantitative analysis of pridopidine's effect on the percentage of innervated NMJs. Data are shown as mean±SEM (n=number of NMJs). *p value <0.05; **p value <0.01; ***p value <0.001 (n=5 mice in each group; Student's t test)

### DETAILED DESCRIPTION OF THE INVENTION

The invention provides for a method for treating a subject afflicted with amyotrophic lateral sclerosis (ALS), comprising periodically administering to the subject an amount of pridopidine effective to treat the human subject.

The invention further provides a method for maintaining, improving, or lessening the decline of ALS patient's functionality, respiratory function, muscle strength, bulbar function, speech or any combination thereof in a subject afflicted with amyotrophic lateral sclerosis (ALS), comprising administering to the subject a therapeutically acceptable amount of pridopidine.

In an embodiment of the invention, the ALS is sporadic ALS.

In an embodiment of the invention, the ALS is familial ALS (FALS). In some embodiments the ALS is juvenile ALS (JALS).

In some embodiments the ALS is not FALS. In some embodiments the ALS is not juvenile ALS (JALS).

In an embodiment of the invention, the type of ALS is classic, bulbar, flail arm, flail leg, pyramidal and respiratory ALS, progressive muscular atrophy, primary lateral sclerosis or progressive bulbar palsy.

In an embodiment of the invention, the subject carries a mutant version of a gene that causes or contributes to ALS pathogenesis. In some embodiments the mutant version of the gene is selected from the group of genes consisting of the superoxide dismutase 1 (SOD1), TAR DNA-binding protein (TARDBP) encoding TDP-43, fused in sarcoma (FUS), p62 (SQSTM1), ubiliquin-2 (UBQLN2), TANK-binding kinase 1 (TBK1), profilin 1 (PFN1), VCP or p97 (VCP), angiogenin (ANG), optineurin (OPTN), C9orf72₂ Sigma-1 Receptor (S1R), Tubulin alpha-4A (TUBA4A), Dynactin (DCTN1) hnRNPA1 (HNRNPA1), Matrin 3(MATR3), Coiled-coil-helix-coiled-coil-helix domain containing 10 (CHCHD10) genes and any combination thereof.

In some embodiments of the invention, maintaining, improving, or lessening the decline of ALS patient's functionality comprises maintaining, improving, or lessening the decline of speech, salivation, swallowing, handwriting, cutting food and handling utensils, dressing and hygiene, turning in bed and adjusting bed clothes, walking, climbing stairs, dyspnea, orthopnea, respiratory insufficiency or any combination thereof in ALS patients.

In an embodiment of the invention, the change in respiratory function is assessed by slow vital capacity (SVC).

In an embodiment of the invention, the maintaining, improving, or lessening the decline in muscle strength is measured isometrically using hand-held dynamometry (HHD), bilateral Hand Grip or combination thereof.

In an embodiment of the invention, the maintaining, improving, or lessening the decline in bulbar function is measured by the ALSFRS-R bulbar subdomain (Q1-Q3) score.

In an embodiment of the invention, the maintaining, improving, or lessening the decline in bulbar function is measured by the CM-BFS.

In an embodiment of the invention, the subject has bulbar dysfunction.

In an embodiment of the invention, the subject has rapid pre-baseline progression.

In an embodiment of the invention, the amount of pridopidine if effective to change time to first evidence of bulbar dysfunction.

In an embodiment of the invention, the maintaining, improving, or lessening the decline in speech is measured by the ALSFRS-R speech domain score (Q1).

In an embodiment of the invention, the maintaining, improving, or lessening the decline in speech is measured by automated algorithmic assessment of speech collected digitally to detect early changes and tracking progression. In an embodiment of the invention, the maintaining, improving, or lessening the decline is measured by the ALS Functional Rating Scale-Revised (ALSFRS-R).

In an embodiment of the invention, the amount of pridopidine is effective to inhibit or reduce progression of a symptom of the ALS in the subject.

In an embodiment of the invention, the symptom of ALS is a clinical symptom of ALS.

In an embodiment of the invention, the symptom of ALS is muscle weakness and hypotrophy, fasciculations and cramps, spastic hypertonus, hyperreflexia, dysarthria, dysphagia and respiratory weakness, behavioral disturbances, dysexecutive impairment, or frontotemporal dementia.

In an embodiment of the invention, the symptom of ALS is a neuropathological symptom.

In some embodiments, the symptom is bulbar palsy or pseudobulbar affect (PBA).

In an embodiment of the invention, the symptom of ALS is muscle atrophy, loss of motor neurons, loss of anterior horn cells, sclerosis of the spinal cord lateral columns, or gliosis.

In one embodiment, the symptom of ALS is a rate of decline (a) in pulmonary function, (b) in functional disability, or (c) in the ability score for the lower extremities. In an embodiment of the invention, the amount of pridopidine is effective to cause survival of the subject or cause neuroprotection in the subject.

In some embodiments of the invention, treatment of the subject with pridopidine results in a lessened decline or an improvement in the subject, in one or more of the following domains, 1) speech, 2) salivation, 3) swallowing, 4) handwriting, 5) cutting food and handling utensils (with or without gastrostomy), 6) dressing and hygiene, 7) turning in bed and adjusting bed clothes, 8) walking, 9) climbing stairs, 10) breathing, 11) dyspnea, 12) orthopnea, and 13) insufficiency.

In some embodiments, patients are monitored for changes in the above domains using a rating scale, for example the Amyotrophic Lateral Sclerosis Functional Rating Scale (ALSFRS) or revised ALSFRS (ALSFRS-R) and a functional change in a patient is monitored over time.

In some embodiments, pseudobulbar affect (PBA) (as measured by CNS-LS) is monitored in the patients. In some embodiments, the severity and/or frequency of emotional outbursts in subjects experiencing PBA is reduced with pridopidine treatment.

In some embodiments of the invention, use of pridopidine maintains, improves or lessens the decline of in disease severity as measured by the ALS Functional Rating Scale-Revised (ALSFRS-R) in ALS patients and/or ALSAQ-5.

In some embodiments of the invention, use of pridopidine maintains, improves or lessens the decline in respiratory function as assessed by slow vital capacity (SVC) in ALS patients.

In some embodiments of the invention, use of pridopidine maintains, improves or lessens the decline in muscle strength as measured by hand held dynamometry (MID) in ALS patients.

In some embodiments of the invention, use of pridopidine results in maintenance, reduction or less increase in phosphorylated neurofilament heavy chain (pNfH) and neurofilament light chain (NfL) in plasma and CSF in ALS patients.

In some embodiments of the invention, use of pridopidine results in maintenance, reduction or less increase in urinary neurotrophin receptor p75 extracellular domain (p75^{ECD}) in ALS patients.

In some embodiments of the invention, use of pridopidine maintains, improves or lessens the decline in speech characteristics as measured by the slope of change in the CNS-BFS speech subdomain in ALS patients.

Several studies have found that speech features, such as jitter, shimmer, articulatory rate, speaking rate, and pause rate, are affected in ALS. In some embodiments of the invention, use of pridopidine maintains, improves or lessens the decline in speech characteristics as measured by automated algorithmic assessment of speech collected digitally as described in Stegmann, G. et al., 2020 which is incorporated herein by reference.

In some embodiments of the invention, use of pridopidine in ALS patients maintains, improves or lessens the decline in voice characteristics as determined by Aural Analytics set of analyses in ALS patients.

In some embodiments of the invention, use of pridopidine maintains, improves or lessens the decline in cognitive function as measured by the Edinburgh Cognitive and Behavioral ALS Screen (ECAS) in ALS patients.

In some embodiments of the invention, use of pridopidine maintains, improves or lessens the decline in home-based clinical assessments (weekly ALSFRS-R, SVC, pinch strength) in ALS patients.

In some embodiment, use of pridopidine maintains, improves or lessens the decline in bulbar function as measured by the CNS-BFS (Center for Neurologic Study Bulbar Function Scale) and the bulbar sub-domain (Q1-Q3) score of the ALSFRS-R total score in ALS patients.

In some embodiment, use of pridopidine maintains, improves or lessens the decline in muscle strength, as measured isometrically using hand-held dynamometry (HHD) and grip strength in ALS patients.

In some embodiment, use of pridopidine maintains, improves or lessens the decline in bulbar function as measured by the slope of change in the CNS-BFS total score in ALS patients.

In some embodiment, use of pridopidine maintains, improves or lessens the decline in bulbar function as measured by the slope of change in the CNS-BFS total score in ALS patients whose calculated ALSFRS-R slope at baseline (48-ALSFRS-R total score at baseline/time since onset) is equal to or greater than 0.75 pt/month.

In some embodiment, use of pridopidine reduces the percentage of ALS patients who develop bulbar symptoms by 6 months among participants without bulbar symptoms at baseline (as defined as a CNS-BES score <30 at baseline) in the active compared to placebo groups.

In an embodiment of the invention, pridopidine is administered daily.

In an embodiment of the invention, pridopidine is administered more often than once daily.

In an embodiment of the invention, pridopidine is administered twice daily. In an embodiment of the invention, pridopidine is administered thrice daily.

In an embodiment of the invention, pridopidine is administered less often than once daily, for example, on alternate days, three times per week, twice per week or once per week.

In an embodiment of the invention, pridopidine is administered daily, twice a week, three times a week or more often than once daily.

In an embodiment of the invention, pridopidine is administered orally.

In some embodiments, a unit dose of the pharmaceutical composition contains 10-250 mg pridopidine. In some embodiments the composition comprises 45 mg, 67.5 mg, 90 mg, or 112.5 mg of pridopidine.

In an embodiment, between 10-225 mg pridopidine is administered to the patient per day. In another embodiment, between 45-180 mg pridopidine is administered to the patient per day. In another embodiment, 10 mg, 22.5 mg, 45 mg, 67.5, mg, 90 mg, 100 mg, 112.5 mg, 125 mg, 135 mg, 150 mg, or 180 mg pridopidine is administered to the patient per day.

In an embodiment, the pharmaceutical composition is administered twice per day. In another embodiment, an equal amount of the pharmaceutical composition is administered at each administration. In an embodiment, the two doses are administered at least 6 hours apart, at least 7 hours, at least 8 hours, at least 9 hours, at least 10 hours, at least 11 hours apart. In some embodiments, the pharmaceutical composition is administered for at least 12 weeks, at least 20 weeks, at least 24 weeks, at least 26 weeks, at least 52 weeks, or at least 78 weeks.

In an embodiment of the invention, the pridopidine is pridopidine hydrochloride.

In an embodiment of the invention, the subject is a human subject.

The invention also provides for pridopidine for use in treating a human subject afflicted with ALS.

The invention also provides for a pharmaceutical composition for use in treating a human subject afflicted with ALS comprising an effective amount of pridopidine.

The invention further provides a method for the treatment of ALS comprises periodically administering to a subject in need thereof an amount of pridopidine effective to treat the ALS.

In an embodiment, the pharmaceutical composition comprises an amount of pridopidine and an amount of a second compound, for example a compound useful in treating patients with ALS.

In some embodiments, the second compound is riluzole, edaravone, a combination of dextromethorphan and quinidine, laquinimod, sodium phonylbutyrate (PB), tauroursodeoxycholic acid, Zilucoplan, Verdiperstat, CNM-Au8 nanocrystalline gold, IC14 or combination of sodium plyenylbutyrate (PB) and tauroursodeoxycholic acid (i.e.AMX0035).

In an embodiment, the pharmaceutical composition for use in treating a subject afflicted with ALS, comprises pridopidine and a second compound which are prepared to be administered simultaneously or contemporaneously.

In an embodiment, the pharmaceutical composition is in a unit dosage form, useful in treating subject afflicted with ALS, which comprises:
a) an amount of pridopidine;
b) an amount of second compound,
wherein the respective amounts of said second compound and said pridopidine in said composition are effective, upon concomitant administration to said subject of one or more of said unit dosage forms of said composition, to treat the subject. In some embodiments, the second compound is riluzole, edaravone, combination of dextromethorphan/quinidine, sodium pilemlbutyrate (PB), tauroursodeoxycholic acid, Zilucoplan, Verdiperstat, CNM-Au8 nanocrystailine gold, IC14 or combination of sodium phenylbutyrate (PB)/tauroursodeoxycholic acid (i.e.AMX0035).

In an embodiment, the pharmaceutical composition comprises an amount of pridopidine for use in treating a subject afflicted with ALS as an add-on therapy to a second compound which is riluzole.

In another embodiment, the pharmaceutical composition comprises an amount of pridopidine for use in treating a subject afflicted with ALS as an add-on therapy to a second compound which is edaravone. In another embodiment, the pharmaceutical composition comprises an amount of pridopidine for use in treating a subject afflicted with ALS as an add-on therapy to a second compound which is dextromethorphan/quinidine. In another embodiment, the pharmaceutical composition comprises an amount of pridopidine for use in treating a subject afflicted with ALS as an add-on therapy to a second compound sodium phenylbutyrate (PH). In another embodiment, the pharmaceutical composition comprises an amount of pridopidine for use in treating a subject afflicted with ALS as an add-on therapy to a second compound tauroursodeoxycholic acid. In another embodiment, the pharmaceutical composition comprises an amount of pridopidine for use in treating a subject afflicted with ALS as an add-on therapy to combination of sodium phenylbutyrate (PB) and tauroursodeoxycholic acid (i.e.AMX0035). In another embodiment, the pharmaceutical composition comprises an amount of pridopidine for use in treating a subject afflicted with ALS as an add-on therapy to a second compound Zilucoplan. In another embodiment, the pharmaceutical composition comprises an amount of pridopidine for use in treating a subject afflicted with ALS as an add-on therapy to a second compound Verdiperstat. In another embodiment, the pharmaceutical composition comprises an amount of pridopidine for use in treating a subject afflicted with ALS as an add-on therapy to a second compound CNM-Au8 nanocrystalline gold. In another embodiment, the pharmaceutical composition comprises an amount of pridopidine for use in treating a subject afflicted with ALS as an add-on therapy to a second compound IC14.

In an embodiment, the pharmaceutical composition comprises an amount of pridopidine for use in treating a subject afflicted with ALS simultaneously or contemporaneously with a second compound which is riluzole. In another embodiment, the pharmaceutical composition comprises an amount of pridopidine for use in treating a subject afflicted with ALS simultaneously or contemporaneously with a second compound which is edaravone. In another embodiment, the pharmaceutical composition comprises an amount of pridopidine for use in treating a subject afflicted with ALS simultaneously or contemporaneously with a second compound which is dextromethorphaniquinidine, In another embodiment, the pharmaceutical composition comprises an amount of pridopidine for use in treating a subject afflicted with ALS simultaneously or contemporaneously with a second compound which is laquinimod. In another embodiment, the pharmaceutical composition comprises an amount of pridopidine for use in treating a subject afflicted with ALS simultaneously or contemporaneously with a second compound which is sodium phenylbutyrate (PB). In another embodiment, the pharmaceutical composition comprises an amount of pridopidine for use in treating a subject afflicted with ALS simultaneously or contemporaneously with a second compound which is tauroursodeoxycholic acid. In another embodiment, the pharmaceutical composition comprises an amount of pridopidine for use in treating a subject afflicted with ALS simultaneously or contemporaneously with a combination of sodium phenylbutyrate (PB) and tauroursodeoxycholic acid (i.e.AMX0035). In another embodiment, the pharmaceutical composition comprises an amount of pridopidine for use in treating a subject afflicted with ALS simultaneously or contemporaneously with a second compound which is Zilucoplan. In another embodiment, the pharmaceutical composition comprises an amount of pridopidine for use in treating a subject afflicted with ALS simultaneously or contemporaneously with a second compound which is Verdiperstat. In another embodiment, the pharmaceutical composition comprises an amount of pridopidine for use in treating a subject afflicted with ALS simultaneously or contemporaneously with a second compound which is CNM-Au8 nanocrystalline gold. In another embodiment, the pharmaceutical composition comprises an amount of pridopidine for use in treating a subject afflicted with ALS simultaneously or contemporaneously with a second compound which is IC14.

In an embodiment, the pharmaceutical composition comprises an amount of a compound which is riluzole for use in treating a subject afflicted with ALS as an add-on therapy to pridopidine. In another embodiment, the pharmaceutical composition comprises an amount of a compound which is edaravone for use in treating a subject afflicted with ALS as an add-on therapy to pridopidine. In another embodiment, the pharmaceutical composition comprises an amount of a compound which is dextromethorphan/quinidine for use in treating a subject afflicted with ALS as an add-on therapy to pridopidine. In another embodiment, the pharmaceutical composition comprises an amount of a compound which is laquinimod for use in treating a subject afflicted with ALS as an add-on therapy to pridopidine. In another embodiment, the pharmaceutical composition comprises an amount of a compound which is sodium plienylbutyrate (PB) for use in treating a subject afflicted with ALS as an add-on therapy to pridopidine. In another embodiment, the pharmaceutical composition comprises an amount of a compound which is tauroursodeoxycholic acid for use in treating a subject afflicted with ALS as an add-on therapy to pridopidine. In another embodiment, the pharmaceutical composition comprises an amount of a combination of sodium phenylbutyrate (PB) and tauroursodeoxycholic acid for use in treating a subject afflicted with ALS as an add-on therapy to pridopidine. In another embodiment, the pharmaceutical composition comprises an amount of a combination of Zilucoplan for use in treating a subject afflicted with ALS as an add-on therapy to pridopidine. In another embodiment, the pharmaceutical composition comprises an amount of a combination of Verdiperstat, for use in treating a subject afflicted with ALS as an add-on therapy to pridopidine. In another embodiment, the pharmaceutical composition comprises an amount of a combination of CNM-Au8 nanocrystalline gold, for use in treating a subject afflicted with ALS as an add-on therapy to pridopidine. In another embodiment, the pharmaceutical composition comprises an amount of a combination of IC14, for use in treating a subject afflicted with ALS as an add-on therapy to pridopidine.

In an embodiment, the pharmaceutical composition comprises an amount of a compound which is riluzole for use in treating a subject afflicted with ALS simultaneously or contemporaneously with pridopidine. In another embodiment the pharmaceutical composition comprises an amount of a compound which is edaravone for use in treating a subject afflicted with ALS simultaneously or contemporaneously with pridopidine. In another embodiment the pharmaceutical composition comprises an amount of a compound which is dextromethorphan/quinidine for use in treating a subject afflicted with ALS simultaneously or contemporaneously with pridopidine. In another embodiment the pharmaceutical composition comprises an amount of a compound which is sodium phenylbutyrate (PB) for use in treating a subject afflicted with ALS simultaneously or contemporaneously with pridopidine. In another embodiment the pharmaceutical composition comprises an amount of a compound which is tauroursodeoxycholic acid for use in treating a subject afflicted with ALS simultaneously or contemporaneously with pridopidine. In another embodiment the pharmaceutical composition comprises an amount of combination of sodium phenyibutyrate (PB)/tauroursodeoxycholic acid (i.e.AMX0035) for use in treating a subject afflicted with ALS simultaneously or contemporaneously with pridopidine. In another embodiment the pharmaceutical composition comprises an amount of combination of Zilucoplan, for use in treating a subject afflicted with ALS simultaneously or contemporaneously with pridopidine. In another embodiment the pharmaceutical composition comprises an amount of combination of Verdiperstat, for use in treating a subject afflicted with ALS simultaneously or contemporaneously with pridopidine. In another embodiment the pharmaceutical composition comprises an amount of CNM-Au8 nanocrystalline gold for use in treating a subject afflicted with ALS simultaneously or contemporaneously with pridopidine. In another embodiment the pharmaceutical composition comprises an amount of combination of, IC14 for use in treating a subject afflicted with ALS simultaneously or contemporaneously with pridopidine. The invention also provides for a compound which is riluzole for use as an add-on therapy to pridopidine in treating a subject afflicted with ALS.

The invention also provides for a compound which is edaravone for use as an add-on therapy to pridopidine in treating a subject afflicted with ALS.

The invention also provides for a compound which is dextromethorphan/quinidine for use as an add-on therapy to pridopidine in treating a subject afflicted with ALS.

The invention also provides for a compound which is sodium phonylbutyrate (PB) for use as an add-on therapy to pridopidine in treating a subject afflicted with ALS.

The invention also provides for a compound which is tauroursodeoxycholic acid for use as an add-on therapy to pridopidine in treating a subject afflicted with ALS.

The invention also provides for a compound which is Zilucoplan for use as an add-on therapy to pridopidine in treating a subject afflicted with ALS.

The invention also provides for a compound which is Verdiperstat for use as an add-on therapy to pridopidine in treating a subject afflicted with ALS.

The invention also provides for a compound which is CNM-Au8 nanocrystalline gold for use as an add-on therapy to pridopidine in treating a subject afflicted with ALS.

The invention also provides for a compound which is IC14 for use as an add-on therapy to pridopidine in treating a subject afflicted with ALS.

The invention also provides for a combination of sodium phenylbutyrate (PB)/tauroursodeoxycholic acid (i.e.AMX0035) for use as an add-on therapy to pridopidine in treating a subject afflicted with ALS.

The invention also provides for pridopidine for use as an add-on therapy to a compound which is riluzole in treating a subject afflicted with ALS.

The invention also provides for pridopidine for use as an add-on therapy to a compound which is edaravone in treating a subject afflicted with ALS.

The invention also provides for pridopidine for use as an add-on therapy to a compound which is dextromethorphan/quinidine in treating a subject afflicted with ALS.

The invention also provides for pridopidine for use as an add-on therapy to a compound which is laquinimod in treating a subject afflicted with ALS.

The invention also provides for pridopidine for use as an add-on therapy to a compound which is sodium phenylbutyrate (PB) in treating a subject afflicted with ALS.

The invention also provides for pridopidine for use as an add-on therapy to a compound which is tauroursodeoxycholic acid in treating a subject afflicted with ALS.

The invention also provides for pridopidine for use as an add-on therapy to a combination of sodium phenylbutyrate (PB)/tauroursodeoxycholic acid (i.e.AMX0035) in treating a subject afflicted with ALS.

The invention also provides for pridopidine for use as an add-on therapy to a compound which is Zilucoplan in treating a subject afflicted with ALS.

The invention also provides for pridopidine for use as an add-on therapy to a compound which is Verdiperstat in treating a subject afflicted with ALS.

The invention also provides for pridopidine for use as an add-on therapy to a compound which is CNM-Au8 nanocrystalline gold in treating a subject afflicted with ALS.

The invention also provides for pridopidine for use as an add-on therapy to a compound which is IC14 in treating a subject afflicted with ALS.

In an embodiment the add-on therapy is for the treatment, prevention, or alleviation of a symptom of ALS.

The invention also provides for a combination of a compound which is riluzole and pridopidine for use in the treatment, prevention, or alleviation of a symptom of ALS.

The invention also provides for a combination of a compound which is edaravone and pridopidine for use in the treatment, prevention, or alleviation of a symptom of ALS.

The invention also provides for a combination of a compound which is dextromethorphan/quinidine and pridopidine for use in the treatment, prevention, or alleviation of a symptom of ALS.

The invention also provides for a combination of a compound which is sodium phenylbutyrate (PB) and pridopidine for use in the treatment, prevention, or alleviation of a symptom of ALS.

The invention also provides for a combination of a compound which is tauroursodeoxycholic acid and pridopidine for use in the treatment, prevention, or alleviation of a symptom of ALS.

The invention also provides for a combination of combination of sodium phenyibutyrate (PB)/tauroursodeoxycholic acid (i.e.AMX0035) and pridopidine for use in the treatment, prevention, or alleviation of a symptom of ALS.

The invention also provides for a combination of a compound which is Zilucoplan and pridopidine for use in the treatment, prevention, or alleviation of a symptom of ALS.

The invention also provides for a combination of a compound which is Verdiperstat and pridopidine for use in the treatment, prevention, or alleviation of a symptom of ALS.

The invention also provides for a combination of a compound which is Au8 nanocrystalline gold and pridopidine for use in the treatment, prevention, or alleviation of a symptom of ALS.

The invention also provides for a combination of a compound which is IC14 and pridopidine for use in the treatment, prevention, or alleviation of a symptom of ALS.

The invention also provides for the use of pridopidine in the manufacture of a medicament for the treatment of ALS.

The method, use and composition further include decreasing the rate of neurological deterioration in the subject.

In an embodiment, the methods of the present invention further comprise administering to the subject a therapeutically effective amount of a second compound which is riluzole or edaravone.

In an embodiment, the methods of the present invention further comprise administering to the subject a therapeutically effective amount of a second compound which is dextromethorphan/quinidine. In an embodiment, the methods of the present invention further comprise administering to the subject a therapeutically effective amount of a second compound which is sodium phenylbutyrate (PB), or tauroursodeoxycholic acid. In an embodiment, the methods of the present invention further comprise administering to the subject a therapeutically effective amount of a combination of sodium phenylbutyrate (PB)/tauroursodeoxycholic acid (i.e.AMX0035). In an embodiment, the methods of the present invention further comprise administering to the subject a therapeutically effective amount of a second compound which is Zilucoplan. In an embodiment, the methods of the present invention further comprise administering to the subject a therapeutically effective amount of a second compound which is Verdiperstat. In an embodiment, the methods of the present invention further comprise administering to the subject a therapeutically effective amount of a second compound which is Au8 nanocrystalline gold. In an embodiment, the methods of the present invention further comprise administering to the subject a therapeutically effective amount of a second compound which is IC14. In an embodiment, the second compound is riluzole. In another embodiment, the second compound is edaravone. In another embodiment, the second compound is dextromethorphan/quinidine. In another embodiment, the second compound is laquinimod. In is another embodiment, the second compound is sodium phenylbutyrate (PB), or tauroursodeoxycholic acid. In an embodiment, the second compound is Zilucoplan. In an embodiment, the second compound is Verdiperstat. In an embodiment, the second compound is Au8 nanocrystalline gold. In an embodiment, the second compound is IC14.

In an embodiment of the invention, pridopidine and the second compound are administered in one unit. In another embodiment the pridopidine and the second compound are administered in more than one unit.

In an embodiment, the amount of pridopidine and the amount of the second compound are administered simultaneously. In an embodiment, the amount of pridopidine and the amount of the second compound are administered contemporaneously.

In another embodiment, the administration of the second compound precedes the administration of pridopidine. In another embodiment, the administration of pridopidine precedes the administration of the second compound.

In an embodiment, a subject is receiving edaravone therapy prior to initiating pridopidine therapy. In another embodiment, a subject is receiving riluzole prior to initiating pridopidine therapy. In another embodiment, a subject is receiving laquinimod prior to initiating pridopidine therapy. In another embodiment, a subject is receiving dextromethorphaniquinidine prior to initiating pridopidine therapy. In another embodiment, a subject is receiving sodium phenylbutyrate (PB) prior to initiating pridopidine therapy. In another embodiment, a subject is receiving tauroursodeoxycholic acid poor to initiating pridopidine therapy. In another embodiment, a subject is receiving a combination of sodium phenylbutyrate (PB)/tauroursodeoxycholic acid (i.e.AMX0035) prior to initiating pridopidine therapy.

In an embodiment, a subject is receiving Zilucoplan therapy prior to initiating pridopidine therapy. In an embodiment, a subject is receiving Verdiperstat therapy prior to initiating pridopidine therapy. In an embodiment, a subject is receiving CNM-Au8 nanocrystalline gold therapy prior to initiating pridopidine therapy. In an embodiment, a subject is receiving IC14 therapy prior to initiating pridopidine therapy.

In another embodiment, a subject is receiving edaravone therapy for at least 24 weeks, 28 weeks, 48 weeks, or 52 weeks prior to initiating pridopidine therapy. In another embodiment, a subject is receiving riluzole therapy for at least 24 weeks, 28 weeks, 48 weeks, or 52 weeks prior to initiating pridopidine therapy. In another embodiment, a subject is receiving dextromethorphan/quinidine therapy for at least 1 week, 2 weeks, 4 weeks, or 6 weeks prior to initiating pridopidine therapy. In another embodiment, a subject is receiving sodium phenylbutyrate (PB) therapy for at least 1 week, 2 weeks, 4 weeks, or 6 weeks prior to initiating pridopidine therapy. In another embodiment, is a subject is receiving tauroursodeoxycholic acid therapy for at least 1 week, 2 weeks, 4 weeks, or 6 weeks prior to initiating pridopidine therapy. In another embodiment, a subject is receiving combination of sodium phenylbutyrate (PB)/tauroursodeoxycholic acid (i.e.AMX0035) therapy for at least 1 week, 2 weeks, 4 weeks, or 6 weeks prior to initiating pridopidine therapy. In another embodiment, a subject is receiving Zilucoplan therapy for at least 24 weeks, 28 weeks, 48 weeks, or 52 weeks prior to initiating pridopidine therapy. In another embodiment, a subject is receiving Verdiperstat therapy for at least 24 weeks, 28 weeks, 48 weeks, or 52 weeks prior to initiating pridopidine therapy. In another embodiment, a subject is receiving CNM-Au8 nanocrystalline gold. therapy for at least 24 weeks, 28 weeks, 48 weeks, or 52 weeks prior to initiating pridopidine therapy. In another embodiment, a subject is receiving IC14 therapy for at least 24 weeks, 28 weeks, 48 weeks, or 52 weeks prior to initiating pridopidine therapy. In an embodiment, a subject is receiving pridopidine therapy prior to initiating edaravone therapy. In another embodiment, a subject is receiving pridopidine therapy for at least 24 weeks, 28 weeks, 48 weeks, or 52 weeks prior to initiating edaravone therapy.

In an embodiment, a subject is receiving pridopidine therapy prior to initiating riluzole therapy. In another embodiment, a subject is receiving pridopidine therapy for at least 24 weeks, 28 weeks, 48 weeks, or 52 weeks prior to initiating riluzole therapy.

In an embodiment, a subject is receiving pridopidine therapy prior to initiating laquinimod therapy. In another embodiment, a subject is receiving pridopidine therapy for at least 24 weeks, 28 weeks, 48 weeks, or 52 weeks prior to initiating laquinimod therapy.

In an embodiment, a subject is receiving pridopidine therapy prior to initiating dextromethorphan/quinidine therapy. In another embodiment, a subject is receiving pridopidine therapy for at least 24 weeks, 28 weeks, 48 weeks, or 52 weeks prior to initiating dextromethorphan/quinidine therapy.

In an embodiment, a subject is receiving pridopidine therapy prior to initiating sodium phenylbutyrate (PB), tauroursodeoxycholic acid or combination of sodium phenylbutyrate (PB)/tauroursodeoxycholic acid (i.e.AMX0035) therapy. In another embodiment, a subject is receiving pridopidine therapy for at least 24 weeks, 28 weeks, 48 weeks, or 52 weeks prior to initiating sodium phenylbutyrate (PB), tauroursodeonicholic acid or combination of sodium phenyibutyrate (PB)/tauroursodeoxycholic acid (i.e.AMX0035) therapy.

In an embodiment, a subject is receiving pridopidine therapy prior to initiating Zilucoplan therapy.

In another embodiment, a subject is receiving pridopidine therapy for at least 24 weeks, 28 weeks, 48 weeks, or 52 weeks prior to Zilucoplan therapy.

In an embodiment, a subject is receiving pridopidine therapy prior to initiating Verdiperstat. In another embodiment, a subject is receiving pridopidine therapy for at least 24 weeks, 28 weeks, 48 weeks, or 52 weeks prior to initiating Verdiperstat therapy.

In an embodiment, a subject is receiving pridopidine therapy prior to initiating CNM-Au8 nanocrystalline gold therapy. In another embodiment, a subject is receiving pridopidine therapy for at least 24 weeks, 28 weeks, 48 weeks, or 52 weeks prior to initiating CNM-Au8 nanocrystalline gold therapy.

In an embodiment, a subject is receiving pridopidine therapy prior to initiating IC14 therapy. In another embodiment, a subject is receiving pridopidine therapy for at least 24 weeks, 28 weeks, 48 weeks, or 52 weeks prior to initiating IC14 therapy.

In an embodiment, between 0.5 mg to 1.5 mg laquinimod is administered to the patient per day. In another embodiment, 0.5 mg or 1.0 mg laquinimod is administered to the patient per day. In an embodiment, laquinimod is administered orally.

In an embodiment, between 10-200 mg riluzole is administered to the patient per day. In another embodiment, 50 mg, 100 mg, or 200 mg riluzole is administered to the patient per day.

In an embodiment, riluzole is administered orally. In an embodiment dextromethorphanlquinidine is administered orally.

In an embodiment sodium phenylbutyrate (PB) is administered orally. In another embodiment, sodium phenylbutyrate (PB) between 1-10 gr/day is administered to the patient per day. In another embodiment, between 1-5 gr/day, 1-3 gr/day, 4-10 gr/day. In another embodiment, sodium phenylbutyrate (PB) is administered once a day, twice a day or more than twice a day.

In an embodiment tauroursodeoxycholic acid is administered orally. In another embodiment, tauroursodeoxycholic acid between 0.5-3 gr/day is administered to the patient per day. In another embodiment, between 0.5-2 gr/day, 1-3 gr/day. In another embodiment, tauroursodeoxycholic acid is administered once a day, twice a day or more than twice a day.

In an embodiment, AMX0035 is administered orally and is administered to the patient in a therapeutic combination including between 0.5-5 g of sodium phenylbutyrate and between 0.2-5 gr/day of tauroursodeoxycholic acid (TUDCA). In another embodiment 3 gr/day of sodium phenylbutyrate and 1 gr/day tauroursodeoxycholic acid (TUDCA) per day, or 9 gr/day of sodium phenylbutyrate and 2gr/day tauroursodeoxycholic acid (TUDCA) per day. In another embodiment, in a combination including between 1-10 gr/day sodium phenylbutyrate and between 0.5-3 gr/day of tauroursodeoxycholic acid. In another embodiment, AMX0035 is administered once a day, twice a day or more than twice a day.

In an embodiment, between 5-60 mg edaravone is administered to the patient per day. In another embodiment, 30 mg, or 60 mg edaravone is administered to the patient per day.

In an embodiment, edaravone is administered by intravenous infusion. In another embodiment, edaravone is administered once per day for 10 days followed by a 14 day drug-free period. In another embodiment, edaravone is administered once per day for 14 days followed by a 14 day drug-free period.

In an embodiment zilucoplan is administered by subcutaneous injection. In another embodiment, zilucoplan is administered in a daily dose of between 0.05-0.5 mg/kg/day. In another embodiment zilucoplan is administered in a daily dose of between 0.22-0.42 mg/kg/day, 0.1-0.3 mg/kg/day or 0.05-0.2 mg/kg/day.

In an embodiment verdiperstat is administered orally. In another embodiment, verdiperstat is administered in a daily dose between 200-2000 mg/day. In another embodiment verdiperstat is administered in a daily dose of 1200 mg/day, between 200-1000 mg/day, 500-1100 mg/day, 1300-2000 mg/day. In another embodiment, verdiperstat is administered twice a day in a dosage of between 100-1000 mg/bid. In another embodiment, verdiperstat is administered twice a day in a dosage of 600 mg/bid, between 100-500 mg/bid, 250-550 mg/bid or 650-1000 mg/bid. In another embodiment, verdiperstat is administered once a day, twice a day or more than twice a day.

In an embodiment CNM-Au8 nanocrystalline gold is administered orally. In another embodiment, CNM-Au8 nanocrystalline gold is administered in a daily dose between 5-50 mg/day. In another embodiment CNM-Au8 nanocrystalline gold is administered in a daily dose of between 5-10 mg/day, 15-20 mg/day, 15-30 mg/day, 20-30 mg/day. In another embodiment, CNM-Au8 nanocrystalline gold is administered once a day, twice a day or more than twice a day.

In an embodiment IC14 is administered intravenously. In another embodiment, IC14 is administered in a daily dose between 0.2-8 mg/kg/day. In another embodiment IC14 is administered in a daily dose of between 1-4 mg/kg/day, 0.2-4 mg/kg/day, 0.2-0.9 mg/kg/day, 5-8 mg/kg/day. In another embodiment, IC14 is administered once a day, twice a day or more than twice a day.

In an embodiment, each of the amount of the second compound when taken alone, and the amount of pridopidine when taken alone is effective to treat a subject. In another embodiment, either the amount of the second compound when taken alone, the amount of pridopidine when taken alone, is less effective to treat the subject. In another embodiment, either the amount of the second compound when taken alone, the amount of pridopidine when taken alone, is not effective to treat the subject.

In an embodiment, pridopidine is administered adjunctively to the second compound. In another embodiment, the second compound is administered adjunctively to pridopidine.

In an embodiment, a loading dose of an amount different from the intended dose is administered for a period of time at the start of the periodic administration.

In some embodiments the methods of this invention make use of a pharmaceutical composition comprising pridopidine or pharmaceutically acceptable salt thereof and at least one analog thereof and pharmaceutically acceptable salt thereof. In another embodiment, the analog compounds of pridopidine or salts thereof are represented by the following structures of compounds 1-7:

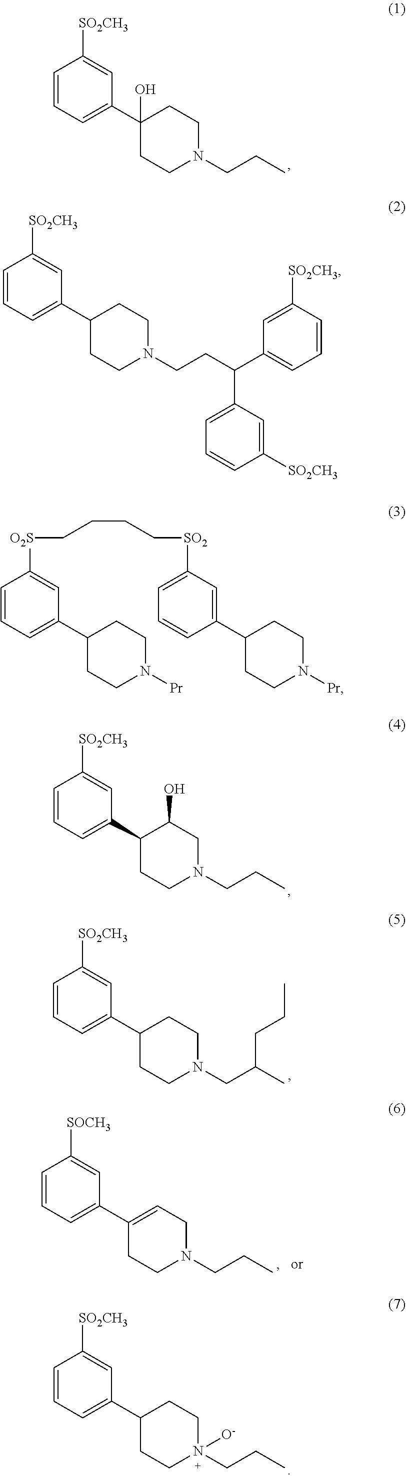

In other embodiments this invention provides a pharmaceutical composition comprising pridopidine or pharmaceutically acceptable salt thereof and compound 1 or pharmaceutically acceptable salt thereof. In other embodiments this invention provides a pharmaceutical composition comprising pridopidine or pharmaceutically acceptable salt thereof and compound 2 or pharmaceutically acceptable salt thereof. In other embodiments this invention provides a pharmaceutical composition comprising pridopidine or pharmaceutically acceptable salt thereof and compound 3 or pharmaceutically acceptable salt thereof. In other embodiments this invention provides a pharmaceutical composition comprising pridopidine or pharmaceutically acceptable salt thereof and compound 4 or pharmaceutically acceptable salt thereof. In other embodiments this invention provides a pharmaceutical composition comprising pridopidine or pharmaceutically acceptable salt thereof and compound 5 or pharmaceutically acceptable salt thereof. In other embodiments this invention provides a pharmaceutical composition comprising pridopidine or pharmaceutically acceptable salt thereof and compound 6 or pharmaceutically acceptable salt thereof. In other embodiments this invention provides a pharmaceutical composition comprising pridopidine or pharmaceutically acceptable salt thereof and compound 7 or pharmaceutically acceptable salt thereof. In other embodiments this invention provides a pharmaceutical composition comprising pridopidine or pharmaceutically acceptable salt thereof and compound 1 and compound 4 or pharmaceutically acceptable salt thereof. In other embodiments, the concentration of compounds 1, 2, 3, 4, 5, 6 or 7 or pharmaceutically acceptable salt thereof within the composition is between 0.001% w/w to 10% w/w. In other embodiments, the concentration of compounds 1, 2, 3, 4, 5, 6 or 7 or pharmaceutically acceptable salt thereof within the composition is between 0.001% w/w to 0.05% w/w. In other embodiments, the concentration of compounds 1, 2, 3, 4, 5, 6 or 7 or pharmaceutically acceptable salt thereof within the composition is between 0.001% w/w to 0.5% w/w. In other embodiments, the concentration of compounds 1, 2, 3, 4, 5, 6 or 7 or pharmaceutically acceptable salt thereof within the composition is between 0.001% w/w to 0.15% w/w. In other embodiments, the concentration of compounds 1, 2, 3, 4, 5, 6 or 7 or pharmaceutically acceptable salt thereof within the composition is between 0.01% w/w to 0.15% w/w. In other embodiments, the concentration of compounds 1, 2, 3, 4, 5, 6 or 7 or pharmaceutically acceptable salt thereof within the composition is between 0.01% w/w to 0.5% w/w. In other embodiments, the concentration of compounds 1, 2, 3, 4, 5, 6 or 7 or pharmaceutically acceptable salt thereof within the composition is between 0.01% w/w to 1% w/w.

In an embodiment provided is a method of enhancing BDNF axonal transport in motor neurons in a subject afflicted with ALS comprising administering to the subject an amount of pridopidine effective to enhance BDNF axonal transport in the subject's motor neurons.

In an embodiment provided is a method of enhancing ERK activation in motor neurons of a subject afflicted with ALS comprising administering to the subject an amount of pridopidine effective to enhance ERK activation in the subject's motor neurons.

In an embodiment provided is a method of preserving neuromuscular junction (NMJ) structure in muscle cells of a subject afflicted with ALS comprising administering to the subject an amount of pridopidine effective to preserving neuromuscular junction structure in the subject's muscles.

Further provided is a method of improving muscle contraction in a subject afflicted with ALS comprising administering to the subject an amount of pridopidine effective to improve muscle contraction function in the subject.

Further provided is a method of improving innervation rate of muscle tissue in a subject afflicted with ALS comprising administering to the subject an amount of pridopidine effective to improve the innervation rate in the subject.

In an embodiment, provided is a method of enhancing motor neuron axonal growth in a subject afflicted with ALS comprising administering to the subject an amount of pridopidine effective to enhance motor neuron axonal growth in the subject.

In an embodiment, provided is a method of enhancing muscle cell survival in a subject afflicted with ALS comprising administering to the subject an amount of pridopidine effective to enhancing muscle cell survival in the subject.

In an embodiment, provided is a method of reducing progression of muscle fiber wasting in a subject afflicted with ALS comprising administering to the subject an amount of pridopidine effective to reduce progression of muscle fiber wasting in the subject.

In an embodiment, provided is a method of reducing axonal degeneration in a subject afflicted. with ALS comprising administering to the subject an amount of pridopidine effective to reduce axonal degeneration in the subject.

In an embodiment, provided is a method of preserving NMJ formation in a subject afflicted with ALS comprising administering to the subject an amount of pridopidine effective to preserve NMJ formation in the subject.

In an embodiment, provided is a method of preserving NMJ structure and function in a subject afflicted with ALS comprising administering to the subject an amount of pridopidine effective to preserve NMJ structure and function in the subject.

In an embodiment, provided is a method of reducing protein aggregation in a subject afflicted with ALS comprising administering to the subject an amount of pridopidine effective to reduce protein aggregation in the subject.

In an embodiment, provided is a method of attenuating pseudobulbar disease progression in a subject afflicted with ALS comprising administering to the subject an amount of pridopidine effective to attenuate pseudobulbar disease progression in the subject.

The invention also provides for a package comprising:
a pharmaceutical composition comprising an amount of pridopidine; and
optionally instructions for use of the pharmaceutical composition to treat a subject afflicted with ALS.

The invention also provides for a therapeutic package for dispensing to, or for use in dispensing to, a subject, which comprises:
one or more unit doses, each such unit dose comprising an amount of pridopidine thereof, wherein the amount of said pridopidine in said unit dose is effective, upon administration to said subject, to treat ALS in the subject, and
a finished pharmaceutical container therefor, said container containing said unit dose or unit doses, said container optionally further containing or comprising labeling directing the use of said package in the treatment of a subject afflicted with ALS. Such unit dose contains 10-250 mg pridopidine, or for example 10 mg, 22.5 mg, 45 mg, 67.5 mg, 90 mg, or 112.5 mg pridopidine.

The invention also provides for a package comprising:
a first pharmaceutical composition comprising an amount of pridopidine and a pharmaceutically acceptable carrier;
a second pharmaceutical composition comprising an amount of a second compound which is riluzole, edaravone, combination of dextromethorphan/quinidine, sodium phenylbutyrate (PB), tauroursodeoxycholic acid or a combination of sodium phenylbutyrate (PB)/tauroursodeoxycholic acid (i.e.AMX0035), Zilucoplan, Verdiperstat, CNM-Au8 nanocrystalline gold, IC1 and a pharmaceutically acceptable carrier; and
optionally instructions for use of the first and second pharmaceutical compositions together to treat a subject afflicted with ALS.

In an embodiment, the amount of the second compound and the amount of pridopidine are prepared to be administered simultaneously or contemporaneously.

The invention also provides for a therapeutic package for dispensing to, or for use in dispensing to, a subject afflicted with ALS, which comprises:
one or more unit doses, each such unit dose comprising:
an amount of pridopidine and
an amount of a second compound which is riluzole, or edaravone, combination of dextromethorphan/quinidine, sodium phenylbutyrate (PB), tauroursodeoxycholic acid, a combination of sodium phenylbutyrate (PB)/tauroursodeoxycholic acid (i.e.AMX0035), Zilucoplan, Verdiperstat, CNM-Au8 nanocrystalline gold or IC14 wherein the respective amounts of said pridopidine and the second compound in said unit dose are effective, upon concomitant administration to said subject, to treat the subject, and
a finished pharmaceutical container therefor, said container containing said unit dose or unit doses, said container optionally further containing or comprising labeling directing the use of said package in the treatment of said subject.

The invention further provides a method for combination therapy for treatment of ALS comprising administering to a subject in need thereof a therapeutically effective amount of pridopidine and therapeutically effective amount of riluzole, edaravone, combination of dextromethorphan/quinidine, sodium phenylbutyrate (PB), tauroursodeoxycholic acid, a combination of sodium phenylbutyrate (PB)/tauroursodeoxycholic acid AMX0035), Zilucoplan, Verdiperstat, CNM-Au8 nanocrystalline gold or IC14.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

All combinations, sub-combinations, and permutations of the various elements of the methods and uses described herein are envisaged and are within the scope of the invention.

### Pharmaceutically Acceptable Salts

As used herein, “pridopidine” means pridopidine base or a pharmaceutically acceptable salt thereof, as well as derivatives, for example deuterium-enriched version of pridopidine and salts. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference. In certain embodiments, pridopidine is a pharmaceutically acceptable salt, such as the HCl salt or tartrate salt. Preferably, in any embodiments of the invention as described herein, the pridopidine is in the form of its hydrochloride salt.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methane sulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials

### Pharmaceutical Compositions

While the pridopidine for use according to the invention may be administered in the form of the raw compound, preferred administration of pridopidine, optionally in the form of a physiologically acceptable salt, is in a pharmaceutical composition together with one or more adjuvants, excipients, carriers, buffers, diluents, and/or other customary pharmaceutical auxiliaries.

In an embodiment, the invention provides pharmaceutical compositions comprising the pridopidine or pharmaceutically acceptable salts or derivatives thereof, together with one or more pharmaceutically acceptable carriers therefore, and, optionally, other therapeutic and/or prophylactic ingredients known and used in the art including, but not limited to, riluzole, edaravone Nuedexta® (dextromethorphan/quinidine), sodium phenylbutyrate (PB), tauroursodeoxycholic acid, a combination of sodium phenylbutyrate (PB)/tauroursodeoxycholic acid (i.e.AMX0035), Zilucoplan, Verdiperstat, CNM-Au8 nanocrystalline gold or IC14.

The carrier(s) must be “acceptable” in the sense of being compatible with the other ingredients of the formulation and suitable for administration to a human subject.

### Combination Therapy

When the invention comprises a combination of the active compound and an additional one, or more, therapeutic and/or prophylactic ingredients, the combination of the invention may be formulated for its simultaneous or contemporaneous administration, with at least a pharmaceutically acceptable carrier, additive, adjuvant or vehicle. This has the implication that the combination of the two active compounds may be administered:
as a combination that is part of the same medicament formulation, the two active compounds being then administered simultaneously, or
as a combination of two units, each with one of the active substances giving rise to the possibility of simultaneous or contemporaneous administration.

The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999).

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug.

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

In one example, combined administration of GA and interferon (IFN) has been experimentally shown to abrogate the clinical effectiveness of either therapy (Brod 2000). In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

### Administration

The pharmaceutical composition of the invention may be administered by any convenient route, which suits the desired therapy. Preferred routes of administration include oral administration, in particular in tablet, in capsule, in dragée, in powder, or in liquid form, intranasal administration, intradermal administration, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds); Modern Pharmaceutics Drugs and the Pharmaceutical Sciences, Vol. 40 (Gilbert S. Banker, Christopher T. Rhodes, Eds). These references in their entireties are hereby incorporated by reference into this application.

### Terms

As used herein, and unless stated otherwise, ach of the following terms shall have the definition set forth below.

As used herein, “riluzole” means riluzole or a pharmaceutically acceptable salt thereof, as well as derivatives, for example deuterium-enriched version of riluzole and salts. Riluzole is descried in Prescribers' Digital Reference which is hereby incorporated by reference (Riluzole PDR 2017).

As used herein, “edaravone” means edaravone or a pharmaceutically acceptable salt thereof, as well as derivatives, for example deuterium-enriched version of edaravone and salts, Edaravone is descried in Prescribers' Digital Reference which is hereby incorporated by reference (Edaravone PDR 2017).

As used herein, “AMX0035” means an oral combination of two drugs already in use, sodium phenylbutyrate (PB) and tauroursodeoxycholic acid (TUDCA). AMX0035 is a combination therapy designed to reduce neuronal death through blockade of key cellular death pathways originating in the mitochondria and endoplasmic reticulum (ER).

A “combination of dextromethorphan and quinidine” or “dextromethorphan/quinidine” or “dextromethorphan hydrobromide/quinidine sulfate” refers to a combination of dextromethorphan hydrobromide (20 mg) and quinidine sulfate (10 mg) such as Nuedexta®. Nuedexta® is a drug currently on the market for treating pseudobulbar affect (PBA) in, inter alia, ALS patients. Nuedexta® has been shown to act on sigma-1 and NMDA receptors in the brain. Recent data demonstrate that the combination has an effect on bulbar function in ALS, but not on other aspects of motor functions (Smith 2017). Dextromethorphan hydrobromide/quinidine sulfate is descried in Prescribers' Digital Reference which is hereby incorporated by reference (Dextromethorphan hydrobromide/quinidine sulfate PDR 2017).

As used herein, an “amount” or “dose” of pridopidine as measured in milligrams refers to the milligrams of underivatized pridopidine base present in a preparation, regardless of the form of the preparation. A “dose of 45 mg pridopidine” means the amount of pridopidine in a preparation is sufficient to provide 45 mg of underivatized pridopidine base having a naturally occurring isotope distribution, regardless of the form of the preparation. Thus, when in the form of a salt, e.g. a pridopidine hydrochloride, the mass of the salt form necessary to provide a dose of 45 mg underivatized pridopidine base would be greater than 45 mg due to the presence of the additional salt ion. Similarly, when in the form of a deuterium-enriched derivative, the mass of the derivatized form necessary to provide a dose of 45 mg underivatized pridopidine base having a naturally occurring isotope distribution would be greater than 45 mg due to the presence of the additional deuterium.

By any range disclosed herein, it is meant that all hundredth, tenth and integer unit amounts within the range are specifically disclosed as part of the invention. Thus, for example, 0.01 mg to 50 mg means that 0.02, 0.03, . . . 0.09; 0.1; 0.2 . . . 0.9; and 1, 2 . . . 49 mg unit amounts are included as embodiments of this invention. By any range of time disclosed herein (i.e. weeks, months, or years), it is meant that all lengths of time of days and/or weeks within the range are specifically disclosed as part of the invention. Thus, for example, 3-6 months means that 3 months and 1 day, 3 months and 1 week, and 4 months are included as embodiments of the invention.

As used herein, “about” in the context of a numerical value or range means ±10% of the numerical value or range recited or claimed.

As used herein, “monotherapy” means treatment with a single active agent, for example treatment with pridopidine alone.

As used herein, “adjunctively” means treatment with or administration of an additional compound, with a primary compound, for example for increasing the efficacy or safety of the primary compound or for facilitating its activity.

As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the pridopidine and a second compound (for example, riluzole). In this case, the combination may be the admixture or separate containers of the pridopidine and the second compound that are combined just prior to administration. Contemporaneous administration, or concomitant administration refer to the separate administration of the pridopidine and the second compound (for example, riluzole) at the same time, or at times sufficiently close together that an additive or preferably synergistic activity relative to the activity of either the pridopidine or the second compound alone is observed or in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding pridopidine therapy to a patient already receiving riluzole therapy.

As used herein, “effective” when referring to an amount of pridopidine refers to the quantity of pridopidine that is sufficient to yield a desired therapeutic response. In a preferred embodiment, the quantity of pridopidine administered does not result in adverse side-effects (such as toxicity, irritation, or allergic response).

“Administering to the subject” or “administering to the (human) patient” means the giving of, dispensing of, or application of medicines, drugs, or remedies to a subject/patient to relieve, cure, or reduce the symptoms associated with a disease, disorder or condition, e.g., a pathological condition.

“Treating” as used herein encompasses inducing inhibition, regression, or stasis of a disease or disorder, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

As used herein, “a subject afflicted with” a disease, disorder or condition means a subject who as been clinically diagnosed to have the disease, disorder or condition.

Glial cell-derived neurotrophic factor (GDNF) is a protein encoded by the GDNF gene and is believed to promote the survival of many types of neurons them.

Brain-derived neurotrophic factor (BDNF), is a protein produced by neurons and serves to keep functioning and to promote the growth of neurons and neurogenesis.

### ALS Functional Rating Scale—Revised (ALSFRS-R)

ALSFRS-R is a quickly administered (5 minutes) ordinal rating scale used to determine participants' assessment of their capability and independence in 12 functional activities. Each functional activity is rated 0-4 for a total score that ranges from 0 to 48, Higher scores indicate better function. Initial validity in ALS patients was established by documenting that, change in ALSFRS-R scores correlated with change in strength over time, was closely associated with quality of life measures, and predicted survival. The test-retest reliability is greater than 0.88 for all test items. The advantages of the ALSFRS-R are that all 12 functional activities are relevant to ALS, it is a sensitive and reliable tool for assessing activities of daily living function in those with ALS, and it is quickly administered. With appropriate training the ALSFRS-R can be administered with high inter-rater reliability and test-retest reliability. The ALSFRS-R can be administered by phone with good inter-rater and test-retest reliability. The equivalency of phone versus in-person testing, and the equivalency of study participant versus caregiver responses have also been established. Additionally, the ALSFRS-R can also be obtained using a web-based interface with good concordance with in-person assessment. All ALSFRS-R evaluators must be NEALS certified.

### Slow Vital Capacity, SVC

The vital capacity (VC) will be determined using the upright slow VC method. All VC evaluators must be NEALS certified. The VC will be measured using the Easyone Air spirometer, and assessments will be performed using a face mask. A printout from the spirometer of all VC trials will be retained. Three VC trials are required for each testing session, however up to 5 trials may be performed if the variability between the highest and second highest VC is 10% or greater for the first 3 trials. Only the 3 best trials are recorded on the CRF. The highest VC recorded is utilized for eligibility. At least 3 measurable VC trials must be completed to score VC for all visits after screening. Predicted VC values and percent-predicted VC values will be calculated using the Quanjer Global Lung Initiative equations.

### Measures of Muscle Strength

Hand Held Dynamometry: HHD will be used as a quantitative measure of muscle strength for this study. Six proximal muscle groups will be examined bilaterally in both upper and lower extremities (shoulder flexion, elbow flexion, elbow extension, hip flexion, knee flexion, and knee extension), all of which have been validated against maximum voluntary isometric contraction (MVIC) testing 19. In addition, wrist extension, abductor pollicis brevis, abductor digiti minimi, first dorsal interosseous contraction and ankle dorsiflexion will be measured bilaterally; these muscles are often affected in ALS.

Bilateral Hand Grip: Bilateral hand grip will be measured using a Jamar hand dynamometer to test the maximum isometric strength of the hand and forearm muscles, measured in pounds.

### Voice Analysis

In addition to the scheduled in clinic voice recordings, voice samples are collected twice per week and at each in person visit, using an app installed on either an android or iOS-based smartphone. The app characterizes ambient noise, then asks participants to perform a set of speaking tasks: reading sentences—5 fixed and 5 chosen at random from a large sentence bank—repeating a consonant-vowel sequence, producing a sustained phonation, and counting on a single breath. Voice signals are uploaded to a HIPAA-compliant web server, where an AI-based analysis identifies relevant vocal attributes. Quality control (QC) of individual samples will occur by evaluation of voice records by trained personnel.

### Center for Neurologic Study Bulbar Function Scale

The Center for Neurologic Study Bulbar Function Scale (CNS-BFS) is a participant self-report scale that has been developed for use as an endpoint in clinical trials and as a clinical measure for evaluating and following ALS patients (Smith et al, 2018). The CNS-BFS consists of three domains (swallowing, speech, and salivation), which are assessed with a 21-question, self-report questionnaire. Higher scores indicate greater bulbar dysfunction.

Participants will be handed the questionnaire and asked to write their answers themselves. Caregivers can also help, if needed.

Instructions on administering the questionnaire during a phone or telemedicine visit will be included in the MOP.

### CNS-Lability Scale

The Center for Neurologic Study Lability Scale (CNS-LS) is a participant self-report scale that has been developed for use as an endpoint in clinical trials and as a clinical measure for evaluating emotional lability. The CNS-LS is a short (seven-question), self-report questionnaire, designed to be completed by the participant, that provides a quantitative measure of the perceived frequency of PBA episodes. Higher scores indicate greater emotional lability. A CNS-LS score of 13 or higher may suggest PBA.

For all in person visits, participants will be handed the questionnaire and asked to write their answers themselves. Caregivers can also help, if needed. During telephone visits, site staff will administer and record data for this scale.

### ALSAQ-40

The Amyotrophic Lateral Sclerosis Assessment Questionnaire-40 (ALSAQ-40) is a participant is self-report health status patient-reported outcome. The ALSAQ-40 consists of forty questions that are specifically used to measure the subjective well-being of participants with ALS and motor neuron disease. Higher scores indicate a decrease in quality of life.

Participants will be handed the questionnaire and asked to write their answers themselves. Caregivers can also help, if needed.

The following numbered clauses define various aspects and features of the present invention:
1. A method for treating a subject afflicted with amyotrophic lateral sclerosis (ALS), comprising periodically administering to the subject an amount of pridopidine effective to treat the subject.
2. The method of clause 1, wherein the amount of pridopidine is effective to inhibit or reduce progression of a symptom of the ALS in the subject.
3. The method of clause 2, where in the symptom is muscles stiffness, muscle weakness, muscle wasting, muscle cramps, difficulty speaking, difficulty swallowing, difficulty breathing, difficulty chewing, difficulty walking, fasciculations, and/or worsening posture.
4. The method of any one of clauses 1-3, wherein the ALS is sporadic ALS.
5. The method of any one of clauses 1-4, wherein the amount of pridopidine is administered daily or wherein the amount of pridopidine is administered more often than once daily.
6. The method of any one of clauses 1-4, wherein the amount of pridopidine is administered twice daily.
7. The method of any one of clauses 1-4, wherein the amount of pridopidine is administered less often than once daily.
8. The method of any one of clauses 1-7, wherein the amount of pridopidine is administered orally.
9. The method of any one of clauses 1-8, wherein the amount of pridopidine administered is from 22.5 mg per day to 225 mg per day.
10. The method of clause 9, wherein the amount of pridopidine administered is from 45 mg per day to 180 mg per day.
11.The method of clause 9, wherein the amount of pridopidine administered is 5 mg, 10 mg, 22.5 mg, 45 mg, 67.5, mg, 90 mg, 100 mg, 112.5 mg, 125 mg, 135 mg, 150 mg, or 180 mg per day.
12. The method of any one of clauses 1-11, wherein the periodic administration continues for at least 24 weeks.
13. The method of any one of clauses 1-12, wherein the pridopidine is pridopidine hydrochloride.
14. The method of any one of clauses 1-13, wherein the subject is a human subject.
15. The method of any one of clauses 1-14, further comprising administering to the subject a therapeutically effective amount of a second compound.
16. The method of clause 15, wherein the second compound is riluzole, edaravone, dextromethorphan/quinidine, sodium phenylbutyrate (PB), tauroursodeoxycholic acid, sodium phenyibutyrate (PB)/tauroursodeoxycholic acid (i.e.AMX0035), Zilucoplan, Verdiperstat, CNM-Au8 nanocrystalline gold or IC14.
17. The method of clauses 15-16, wherein the pridopidine and the second compound are administered in one unit.
18. The method of clauses 15-16, wherein the pridopidine and the second compound are administered in more than one unit.
19. The method of any one of clauses 15-18, wherein the second compound is riluzole.
20. The method of clause 19, wherein 10 mg-200 mg or 50 mg, 100 mg or 200 mg of riluzole is administered to the subject per day.
21. The method of any one of clauses 15-20, wherein the riluzole is administered orally.
22. The method of any one of clauses 15-18, wherein the second compound is edaravone.
23. The method of clause 22, wherein 5-60 mg or 30 mg or 60 mg of edaravone is administered to the subject per day.
24. The method of any one of clauses 15-18 and 22-23, wherein the edaravone is administered by intravenous infusion.
25. The method of any one of clauses 15-18 and 22-24, where the edaravone is administered once per day for 14 days or 10 days followed by a 14 day drug-free period.
26. The method of any one of clauses 15-18, wherein the second compound is dextromethorphan/quinidine.
27. The method of clause 26, wherein 10, 20, or 40 mg of dextromethorphan is administered to the subject per day and 5, 10 or 20 mg of quinidine is administered to the subject per day.
28. The method of any one of clauses 26-27, wherein the dextromethorphan/quinidine is administered orally.
29. The method of any one of clauses 15-28, wherein the amount of pridopidine and the amount of the second compound are administered simultaneously.
30. The method of any one of clauses 15-28, wherein the administration of the second compound substantially precedes the administration of pridopidine.
31. The method of any one of clauses 15-28, wherein the administration of pridopidine substantially precedes the administration of the second compound.
32. The method of any one of clauses 15-28, wherein the subject is receiving edaravone therapy, dextromethorphan/quinidine therapy, riluzole therapy, Zilucoplan therapy, Verdiperstat therapy, CNM-Au8 nanocrystalline gold therapy or IC14 therapy prior to initiating pridopidine therapy.
33. The method of clause 32, wherein the subject is receiving edaravone therapy, dextromethorphan/quinidine therapy, riluzole therapy, Zilucoplan therapy, Verdiperstat therapy, CNM-Au8 nanocrystalline gold therapy or IC14 therapy for at least 24 weeks, 28 weeks, 48 weeks, or 52 weeks prior to initiating pridopidine therapy.
34. The method of any one of clauses 15-28, wherein the subject is receiving pridopidine therapy prior to initiating edaravone therapy, dextromethorphan/quinidine therapy, riluzole therapy Zilucoplan therapy, Verdiperstat therapy, CNM-Au8 nanocrystalline gold therapy or IC14 therapy.
35. The method of clause 34, wherein the subject is receiving pridopidine therapy for at least 24 weeks, 28 weeks, 48 weeks, or 52 weeks prior to initiating edaravone therapy, dextromethorphan/quinidine therapy, riluzole therapy, Zilucoplan therapy, Verdiperstat therapy, CNM-Au8 nanocrystalline gold therapy or IC14 therapy.
36. The method of any one of clauses 15-35, wherein each of the amount of the second compound when taken alone, and the amount of pridopidine when taken alone is effective to treat the subject
37. The method of any one of clauses 15-35 wherein either the amount of the second compound when taken alone, the amount of pridopidine when taken alone, or each such amount when taken alone is not effective to treat the subject.
38. The method of any one of clauses 15-35, wherein either the amount of the second compound when taken alone, the amount of pridopidine when taken alone, or each such amount when taken alone is less effective to treat the subject.
39. The method of any one of clauses 15-38, wherein the pridopidine is administered adjunctively to the second compound.
40. The method of any one of clauses 15-38, wherein the second compound is administered adjunctively to the pridopidine.
41. The method of any one of clauses 1-40, wherein a loading dose of an amount different from the intended dose is administered for a period of time at the start of the periodic administration.
42. A method of enhancing BDNF axonal transport in motor neurons in a subject afflicted with ALS comprising administering to the subject an amount of pridopidine effective to enhance BDNF axonal transport in the subject's motor neurons.
43. A method of improving formation and function in a subject afflicted with ALS comprising administering to the subject an amount of pridopidine effective to improve NMJ formation and muscle contraction function in the subject.
44. A method of improving innervation rate of muscle tissue in a subject afflicted with ALS comprising administering to the subject an amount of pridopidine effective to improve innervation rate in the subject.
45. A method of enhancing motor neuron axonal growth in a subject afflicted with ALS comprising administering to the subject an amount of pridopidine effective to enhance motor neuron axonal growth in the subject.
46. A method of enhancing muscle contraction in a subject afflicted with ALS comprising administering to the subject an amount of pridopidine effective to enhance the muscle contraction in the subject.
47. A method of restoring muscle contraction in a subject afflicted with ALS comprising administering to the subject an amount of pridopidine effective to improve the muscle contraction in the subject.
48. A pharmaceutical composition comprising an effective amount of pridopidine for use in treating a subject afflicted with ALS.
49. Use of an amount of pridopidine for the manufacture of a medicament for use in treating a subject afflicted with ALS.
50. A package comprising:
   a) a pharmaceutical composition comprising an amount of pridopidine; and optionally
   b) instructions for use of the pharmaceutical composition to treat a subject afflicted with ALS.
51. A therapeutic package for dispensing to, or for use in dispensing to, a subject, which comprises:
   a) one or more unit doses, each such unit dose comprising an amount of pridopidine thereof, wherein the amount of said pridopidine in said unit dose is effective, upon administration to said subject, to treat ALS in the subject, and
   b) a finished pharmaceutical container therefor, said container containing said unit dose or unit doses, said container further containing or comprising labeling directing the use of said package in the treatment of a subject afflicted with ALS.
52. A package comprising;
   a) a first pharmaceutical composition comprising an amount of pridopidine and a pharmaceutically acceptable carrier;
   b) a second pharmaceutical composition comprising an amount of a second compound which is riluzole, edaravone, dextromethorphaniquinidine, sodium phenylbutyrate (PB), tauroursodeoxycholic acid, sodium phenyibutyrate (PB)/tauroursodeoxycholic acid (i.e.AMX0035), Verdiperstat, CNM-Au8 nanocrystalline gold or IC14 and a pharmaceutically acceptable carrier; and optionally
   c) instructions for use of the first and second pharmaceutical compositions together to treat a subject afflicted with ALS.
53. The package of clause 52, wherein the amount of the second compound and the amount of pridopidine are prepared to be administered simultaneously or contemporaneously.
54. A therapeutic package for dispensing to, or for use in dispensing to, a subject afflicted with ALS, which comprises:
   a) one or more unit doses, each such unit dose comprising:
   i) an amount of pridopidine and
   ii) an amount of a second compound which is riluzole, edaravone, dextromethorphan/quinidine, Zilucoplan, Verdiperstat, CNM-Au8 nanocrystalline gold or IC14;
   wherein the respective amounts of said pridopidine and the second compound in said unit dose are effective, upon concomitant administration to said subject, to treat the subject, and
   b) a finished pharmaceutical container therefor, said container containing said unit dose or unit doses, said container further containing or comprising labeling directing the use of said package in the treatment of said subject.
55. A pharmaceutical composition comprising an amount of pridopidine and an amount of a second compound which is riluzole, edaravone, dextromethorphan/quinidine, Zilucoplan, Verdiperstat, CNM-Au8 nanocrystalline gold or IC14.
56. The pharmaceutical composition of clause 55 for use in treating a subject afflicted with ALS, wherein the pridopidine and the second compound are prepared to be administered simultaneously, contemporaneously or concomitantly.
57. A pharmaceutical composition in unit dosage form, useful in treating a subject afflicted with ALS, which comprises:
   a) an amount of pridopidine;
   b) an amount of second compound which is riluzole, edaravone, dextromethorphan/quinidine, Zilucoplan, Verdiperstat, CNM-Au8 nanocrystalline gold or IC14,
   wherein the respective amounts of said second compound and said pridopidine in said composition are effective, upon concomitant administration to said subject of one or more of said unit dosage forms of said composition, to treat the subject.
58. A pharmaceutical composition comprising an amount of pridopidine for use in treating a subject afflicted with ALS as an add-on therapy to a second compound which is riluzole, edaravone, Zilucoplan, Verdiperstat, CNM-Au8 nanocrystalline gold, IC14 or dextromethorphan/quinidine.
59. A pharmaceutical composition comprising an amount of pridopidine for use in treating a subject afflicted with ALS simultaneously, contemporaneously or concomitantly with a second compound which is riluzole, edaravone, dextromethorphan/quinidine, Zilucoplan, Verdiperstat, CNM-Au8 nanocrystalline gold or IC14.
60. A pharmaceutical composition comprising an amount of a compound which is riluzole, edaravone or dextromethorphan/quinidine for use in treating a subject afflicted with ALS as an add-on therapy to pridopidine.
61. A pharmaceutical composition comprising an amount of a compound which is riluzole, edaravone or dextromethorphanlquinidine for use in treating a subject afflicted with ALS simultaneously, contemporaneously or concomitantly with pridopidine.
62. A compound which is riluzole, edaravone, dextromethorphan/quinidine, Zilucoplan, Verdiperstat, CNM-Au8 nanocrystalline gold or IC14 for use as an add-on therapy to pridopidine in treating a subject afflicted with ALS.
63. pridopidine for use as an add-on therapy to a compound which is riluzole, edaravone dextromethorphan/quinidine or Zilucoplan, Verdiperstat, CNM-Au8 nanocrystalline gold or IC14 in treating a subject afflicted with ALS.
64. The add-on therapy of clause 63, wherein the therapy is for the treatment, prevention, or alleviation of a symptom of ALS.
65. A combination of pridopidine with a compound which is riluzole, edaravone, dextromethorphan/quinidine, Zilucoplan, Verdiperstat, CNM-Au8 nanocrystalline gold or IC14 for use in the treatment, prevention, or alleviation of a symptom of ALS.

Throughout this application, certain publications and patent application publications are referenced. Full citations for the publications may be found immediately preceding the claims. The disclosures of these publications and patent application publications in their entireties are hereby incorporated by reference into this application in order to describe more fully the state of the art to which this invention relates.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Experiment 1

### Axonal Transport Assays

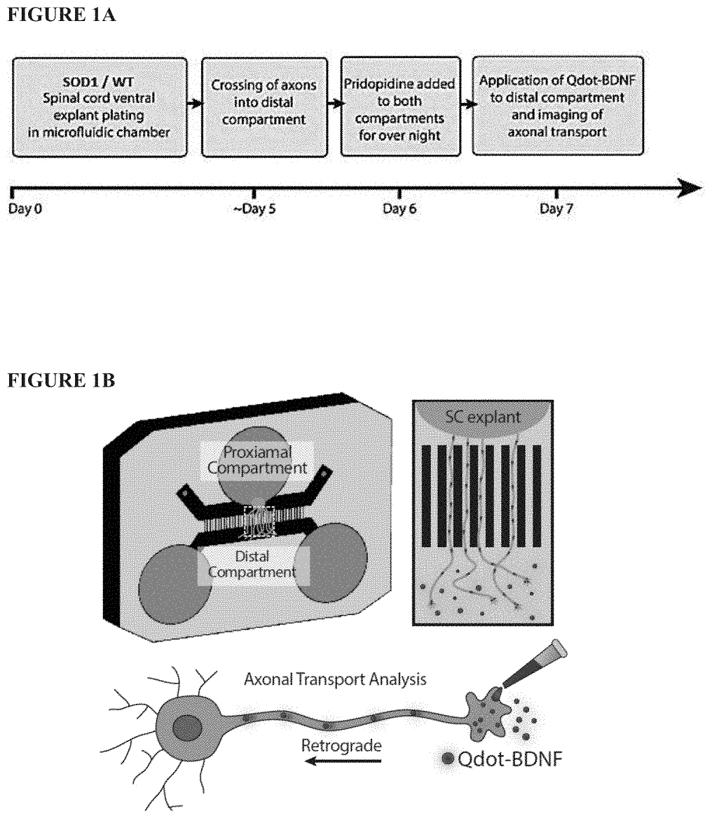

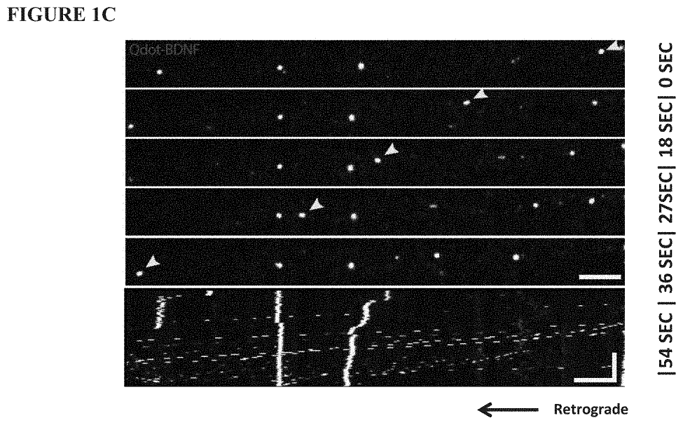

Healthy motor neurons (MN) extend axons over long distances and through varying extracellular microenvironments to form synapses with muscles. The ability of the neuron to maintain this specialized morphology depends on cytoskeletal elements and continuous transport of proteins and organelles to and from the cell body. Cytoskeletal alterations are a major pathway implicated in the pathogenesis of ALS affecting axonal transport, growth and neuromuscular junction (NMJ) function (Eykens and Robberecht, 2015). Alterations in axonal transport are one of the first cellular processes that occur in neurodegenerative disease, including ALS. Axonal transport was evaluated using an in vitro compartmentalized system of microfluidic chambers (MFC) that separates neuronal cell bodies from their axons and synapses. This enables the study of retrograde/anterograde transport by specific monitoring and manipulation of cellular microenvironments (FIG. 1B; Zahavi 2015; Ionescu 2016).

Quantum-Dot labeled BDNF (Qdot BDNF) is retrogradely transported in axons of motor neurons grown from spinal cord explants in a microfluidic chamber (MFC). A MFC was used to analyze Qdot BDNF axonal transport. Axonal transport of BDNF in the SOD1 model (SOD1G93A) for ALS has been studied (Bilsland 2010; Perlson 2009; De Vos 2007). The effect of pridopidine on transport of Qdot BDNF along the axons of motor neurons was assessed in spinal cord explants from E12.5 SOD1 G93A and WT littermate mice (WT). Experimental workflow for the axonal transport assay (from left to right, FIG. 1A): SOD1G93 A or wild Type (WT) spinal cord explants were plated in the MFC. At about 5 days post plating, axons began to cross over into the distal compartment. On day 6 post plating, an amount of pridopidine was added to both compartments.

On day 7, Qdot-BDNF was added to the distal compartment and axonal transport was imaged using a high-resolution spinning-disk confocal microscope. Schematic illustration of microfluidic chamber system (FIG. 1B): Explants planted in the proximal compartment extend axons to the distal compartment, where Qdot-BDNF is applied exclusively prior to visualization.

Spinning disk confocal microscopy was used to track Qdot BDNF along the axons of motor neuron explant cultures. Time lapse images of Qdot-BDNF axonal transport as acquired at 60× magnification (FIG. 1C). Arrowheads point to a single Qdot-BDNF particle that is retrogradely transported (left) towards the cell body. Scale bar: 10 μm. Bottom panel shows a kymograph, which plots distance travelled over time, of a complete Qdot-BDNF time-lapse movie. Scale bars: horizontal 10 μm; vertical 100 seconds (FIG. 1C)

Vehicle and pridopidine were added to both compartments at 2 concentrations (0.1 μM, and 1 μM) on experimental day 6, and Qdot BDNF was added to the distal compartment after overnight incubation with pridopidine (FIGS. 1A and 1B). Six independent biological repeats, from 6 different cultures were tested so that from each culture and explant with neurons/glia ˜250 BDNF particles is were followed along the axons in the grooves. Velocity refers to the movement of a single BDNF particle. The experiment was repeated with MNs from mice in which sigma 1 receptor was genetically deleted (S1R KO or S1R−/−) (Lana, 2003). Ventral spinal cord sections from S1R−/− mice embryos were cultured and plated in the MFC as described above, and the axonal transport of Qdot-BDNF was analyzed.

SOD1G93 and S1R−/− explants with or without pridopidine were compared to wild-type littermate controls (WT).

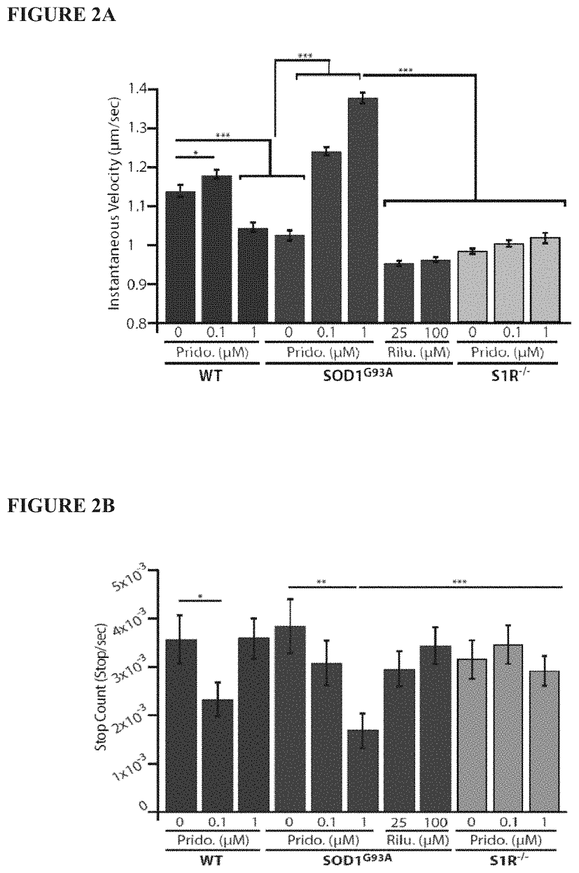

Qdot-BDNF particle tracking was performed on Bitplane Imaris, using the semi-automated spot tracking function, Inclusion criteria for particle analysis: track duration >10 frames; average velocity ≥0.2 μm/sec; stop duration: speed <0.1 μm/sec for 3 frames. Data were then exported to MATLAB for further analysis of particle transport including Instantaneous Velocities (FIG. 2A) from 6 independent cultures; and Stop count (FIG. 2B).

### Results:

FIG. 2A demonstrates that pridopidine enhanced BDNF axonal transport instantaneous velocity in SOD1G934 motor neurons. instantaneous velocity of BDNF retrograde transport is typically reduced in SOD1G93A motor neurons. SOD1G93A MNs show slower velocities vs the WT MNs. Pridopidine treatment accelerated the instantaneous velocities both in WT MNs (0.1 μM) and SOD1G93A MNs (0.1 μM and 1 μM). Application of 25 μM or 100 μM Riluzole to SOD1G93A MNs did not affect the instantaneous velocities. S1R−/− MNs revealed reduced velocity of BDNF axonal transport. Pridopidine at either 0.1 μM or 1 μM was not able to recover these defects in S1R KO MNs (FIG. 2A).

Particle stop count (number of counted stops of Qdot-BDNF per second), shows that pridopidine reduced the number of pauses during axonal transport in WT MNs (0.1 μM only) and SOD1G93A MNs (both 0.1 μM and 1 μM). Axonal transport parameters of Qdot-BDNF in S1R−/− MNs show that they are not responsive to pridopidine at any of the concentrations tested (FIG. 2B).

These results demonstrate that pridopidine enhanced BDNF axonal transport in SOD1G93A motor neurons and has the capacity to correct ALS related deficits.

### Experiment 2

### Axon-Muscle Growth/Degeneration Assays

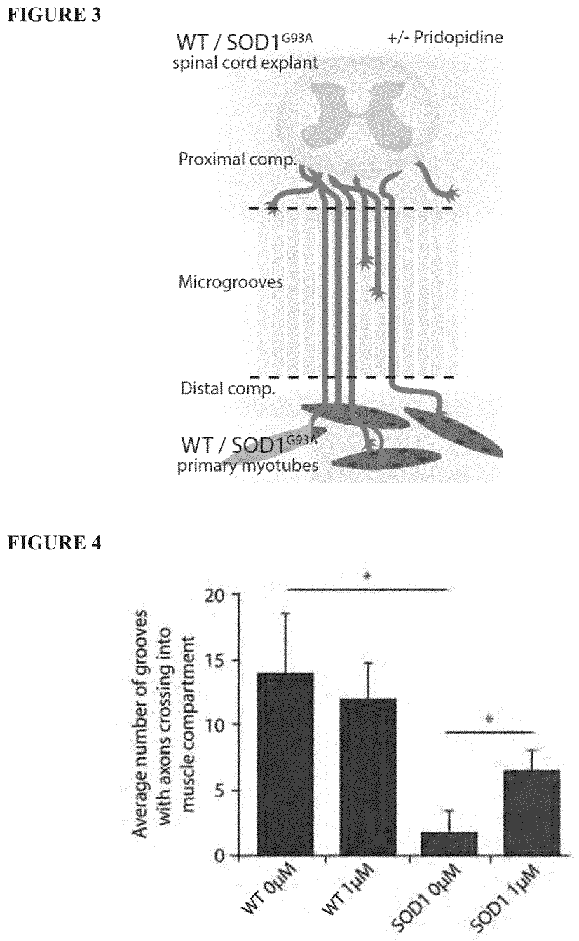

An early event in the pathogenesis of ALS is axonal degeneration. The compartmental co-culture microfluidic chamber system was used to determine whether pridopidine alters axonal degeneration (FIG. 3). Primary muscle cells from presymptomatic (P60) SOD1G93A or WT mice were cultured. On day 6, primary skeletal myoblasts were cultured in the distal compartment of a MFC. About six days later (day 12), ventral spinal cord explants from WT or SOD1G93A E12.5 mouse embryos that express H139-GFP (a specific motor neuron marker) were plated in the proximal compartment, followed by application of pridopidine or vehicle to both compartments. Pridopidine was refreshed every other day. Two days post explant plating (day 14), motor axon growth and degeneration were evaluated using live imaging on a spinning disc confocal system. Axonal growth was tracked by imaging every 10 min for 8 hrs. Experiments were repeated three times.

### Results:

The data demonstrate that pridopidine increased axonal growth (FIG. 4). Myocytes carrying the SO1DG93A mutation have a reduced number of healthy axons that are able to cross into the distal compartment of the microfluidic compartmental chamber as compared with WT (LM) myocytes. Treatment with 1 μM pridopidine (furthest right bar) showed an increased number of axons crossing into the distal compartment (compartment with muscle cells). (Y axis is average number of grooves with axons crossing into muscle compartment).

These results demonstrate that pridopidine enhanced axonal growth and has the capacity to correct ALS related deficits.

### Experiment 3

### Measurement of Neuromuscular Junction (NMJ) Formation and Function

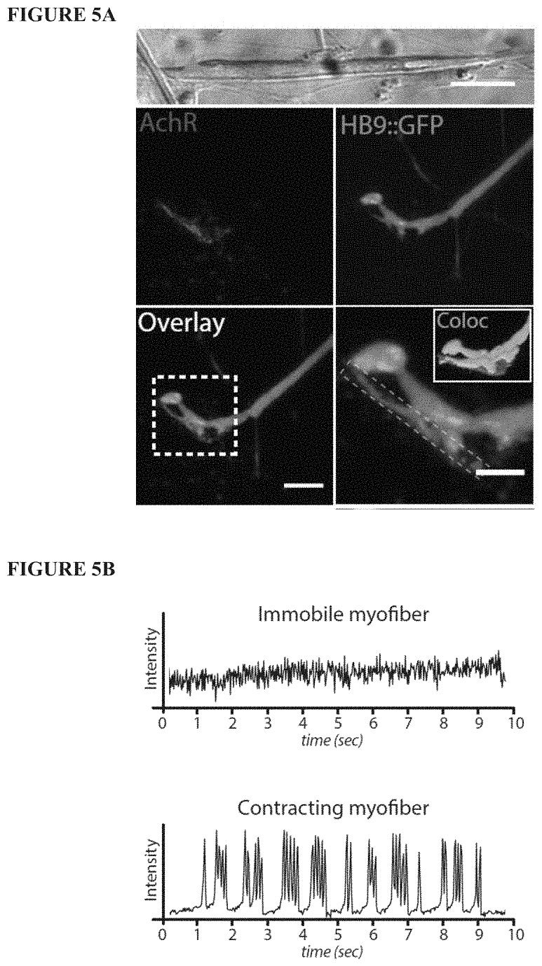

Synapse disruption is the earliest cellular compartment disrupted in ALS. To test the ability of pridopidine to affect synapse function in an ALS model, cultures from Experiment 2 were grown for approximately four additional days (day 18), when axons extend into the distal compartment and generate NMJs. In this co-culture, MN axons formed NMJs on fully differentiated primary myocytes. These can be observed by the co-localization of the post-synaptic marker (AchR, ecetyl choline receptor) with the Hb9:GFP neuronal marker. FIG. 5A: Upper panel: Phase-contrast microscope image of a myocyte in the distal compartment connected by axons (arrowheads). Scale bar: 20 μm. Lower panel: High magnification images of myocyte:MN contact points reveal the formation of NMJs as seen by co-localization of post synaptic AChR with HB9::GFP axons and 3-dimensional co-localization of pre and post-synaptic markers (coloc). To evaluate NMJ function, movies of muscle contraction were acquired at a frame rate of 30 frames per second for 1000 frames (FIG. 5B). Muscle contraction traces as extracted from intensity over time measurements of muscle contraction show the flat trace of a non-contracting, immobile myocyte (upper), and the trace of a contracting myocyte demonstrating multiple bursting events (lower).

To study the effect of pridopidine on MN and NMJ formation and function, either 0.1 or 1 μM pridopidine or vehicle were added. Measurement of % innervation and innervation-induced contraction in myotubes was evaluated using live cell imaging as previously reported (Ionescu 2016; Zahavi 2015). Briefly, contractile activity of muscles in the distal compartment of the MFC, which were overlapped by at least one axon was examined. Muscles were categorized into two groups: ‘Contracting’ or ‘Non-contracting’, depending on their motile activity during the movie. The motility of muscles was validated by generating intensity-over-time plots for each muscle (FIG. 5B). The number of contracting muscle fibers per chamber was divided by the total number of muscle fibers analyzed in the same chamber, yielding the percentage of contracting myotubes as an output for NMJ activity.

### Results:

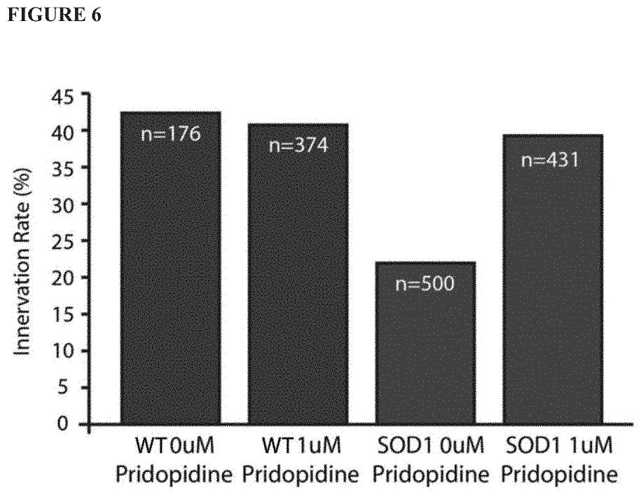

Treatment of muscle cells with pridopidine was able to induce innervation leading to higher likelihood of bursting muscle patterns. Innervation rate of muscles carrying the SOD1 mutation is lower compared to WT (wild type) muscles (20% innervation compared to ˜40% in WTs). Pridopidine at 1 μM increases the innervation rate of muscles carrying SOD1 mutation to near WT levels (FIG. 6).

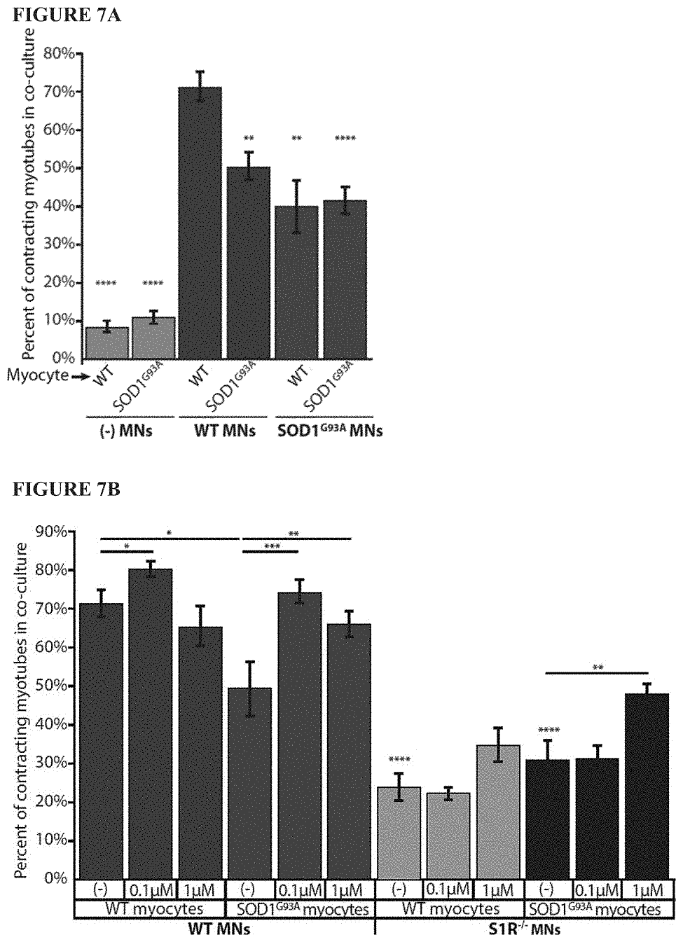

Quantification of the percentage of myofibers shows that in the absence of MNs, only ˜10% of muscles contracted in the absence of MNs versus 74% in cultures including MNs. Co-culture combinations that include at least one of the cell types expressing SOD1G93A show a significantly lower percentage of contracting myocytes (FIG. 7A). Treatment of SOD myocytes co-cultured with pridopidine (0.1 μM and 1 μM) increases the percentage of contracting myocytes and restores neuromuscular activity to WT levels. Combination of S1R−/− MNs with WT myocytes results in a low number of contracting myotubes compared to WT MNs. Application of 0.1 μM pridopidine to S1R−/− co-cultures did not restore the neuromuscular activity, as seen for the same concentration of pridopidine in co-cultures with WT neurons. The increase in the percentage of contracting myocytes observed after treatment with 1 μM pridopidine is significantly lower than the percentage of contracting myocytes in untreated and treated WT cultures. Data are shown as mean±SEM. *p-value <0.05; **p-value <0.01, ***p-value <0.001, ****p-value <0.0001.

### (Student's T-Test)

### Experiment 4

### Activation of ERK in WT and SOD1G93A MNs

The ERK pathway promotes numerous cellular functions including proliferation and differentiation. ERK phosphorylation (activation) in neurons is associated with neurotrophic signaling, such as BDNF, which promotes neuroprotection and neuronal survival (Bonni 1999). It was previously established that pridopidine enhances BDNF signaling in rat striatum through S1R, which in turn, enhances ERK activation (Geva 2016). Primary MN cultures at 2DIV were starved overnight in neurotrophin- and serum-free medium (PNB). The following day, cultures were treated for 30 minutes with pridopidine or with BDNF as a positive control.

### Results:

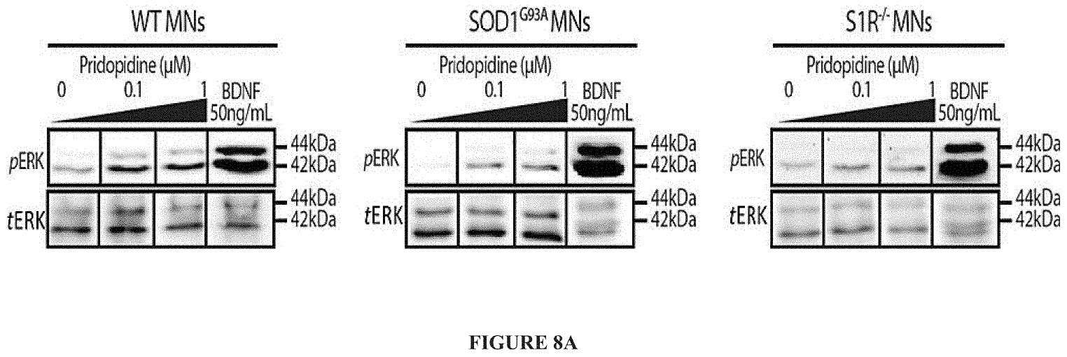

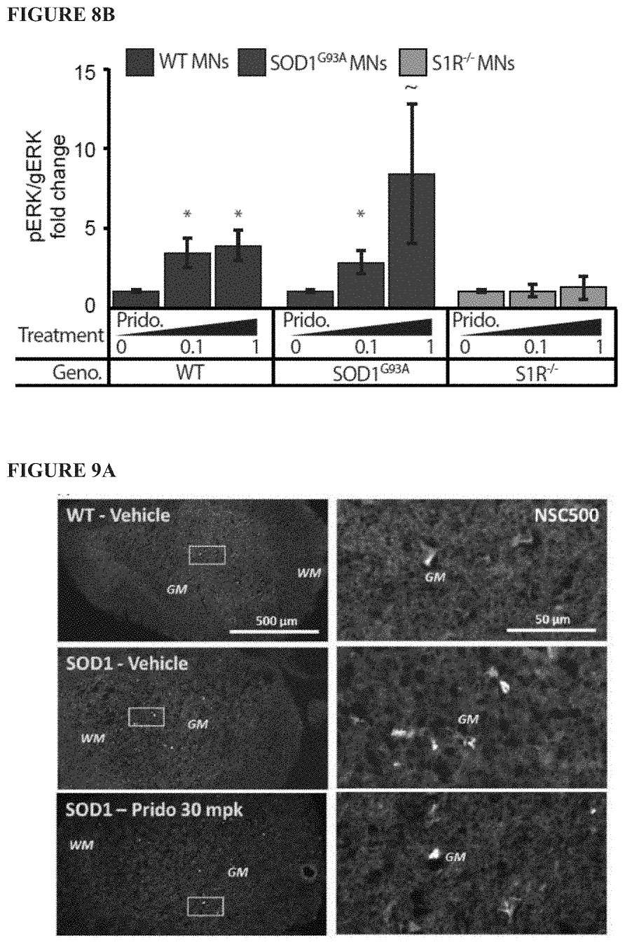

Western blot analyses for phosphorylated ERK (pERK) shows significant activation of ERK by pridopidine (0.1 μM and 1 μM), as early as 30 minutes after application in WT (left panel) and SOD1G93A (middle panel) MN cultures, but not in S1R−/− MN cultures (right panel) (FIG. 8A). Quantification of pERK reveals ˜3.5 and ˜4-fold increase in WT MNs following 0.1 μM and 1 μM pridopidine, respectively. SOD1G93A exhibits ˜2.9 and ˜8.5-fold increase in pERK following 0.1 μM and 1 μM pridopidine, respectively. Data are shown as the mean pERK/ERK ratios±SEM. *p-value <0.05, ˜p-value<0.1 (Student's t-test) (FIG. 8B).

### Experiment 5

### Effect of Pridopidine on Mutant SOD1 Aggregates in the Spinal Cord of SOD1G93A Mice

Pridopidine induced neuroprotective properties by activation of the S1R, as demonstrated for its effect on axonal transport, axonal degeneration, NMJ function and ERK activation. The S1R resides on the ER membrane in close proximity to the mitochondrial outer membrane, where the mutant SOD1 protein tends to aggregate in the spinal cord of SOD1G93A mice (Millecamps and Julien 2013). Pre-symptomatic SOD1G93A mice (5 weeks of age) and WT controls were treated with either saline or 30 mg/kg pridopidine, by daily s.c. (subcutaneous) administration for 11 weeks (until 16 weeks of age). At the end of the experiment, lumbar spinal cords (L1-L6) were extracted, fixed, and embedded for cryosectioning. Next, 10 μM sections were prepared and stained with NSC500 dye to visualize SOD1 aggregates (Hammarström 2010). The in vivo effect of pridopidine treatment on the number of mutant SOD1 aggregates in grey and white matter of spinal cord was evaluated.

### Results:

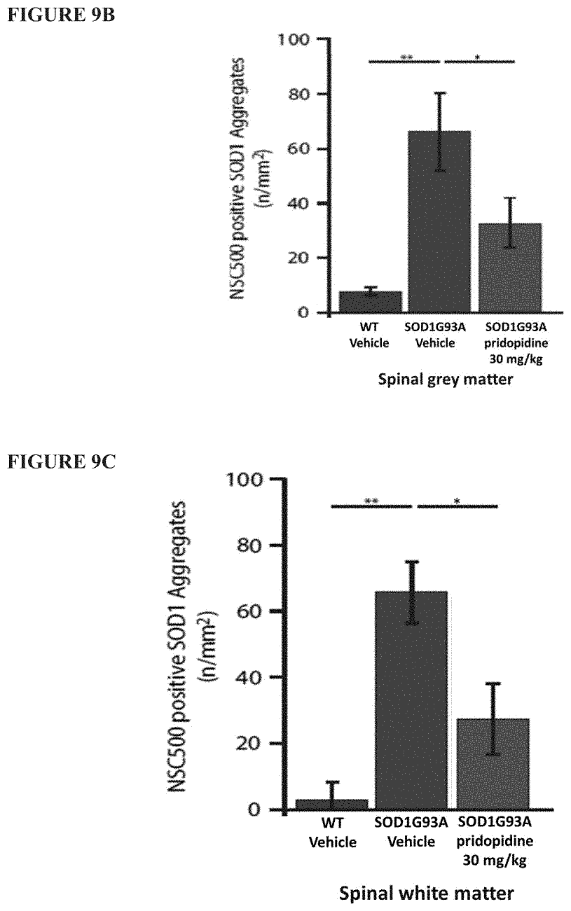

FIG. 9A—Left panel: low magnification representative images of fluorescently labeled spinal cords for 4 mouse groups. Right panel: high magnification images for the regions marked in the left panel by a square. Scale bars: Left panel: 500 μm; Right panel 50 μm. Top to bottom: WT vehicle, SOD1G93A vehicle, SOD1G93A 30 mg/kg, all stained with NSC500 dye to label mutant SOD1 protein aggregates. A significant increase in the number of mSOD1 aggregates was observed in both the gray and white matter of the spinal cords of SOD1G93A mice compared with WI mice. Pridopidine 30 mg/kg significantly reduced the number of aggregates in both the gray and white matters of SOD1G93A spinal cords by ˜50% (FIGS. 9A-9C). Data are shown as the mean±SEM. *p-value <0.05; **p-value <0.01 (one-way ANOVA followed by Fisher's LSD post hoc tests). (FIGS. 9B-9C y-axis is number of NSC500-positive SOD1 aggregates per squared mm).

### Experiment 6

### In-Vivo Evaluation of Muscle Fiber Atrophy and NMJ Preservation

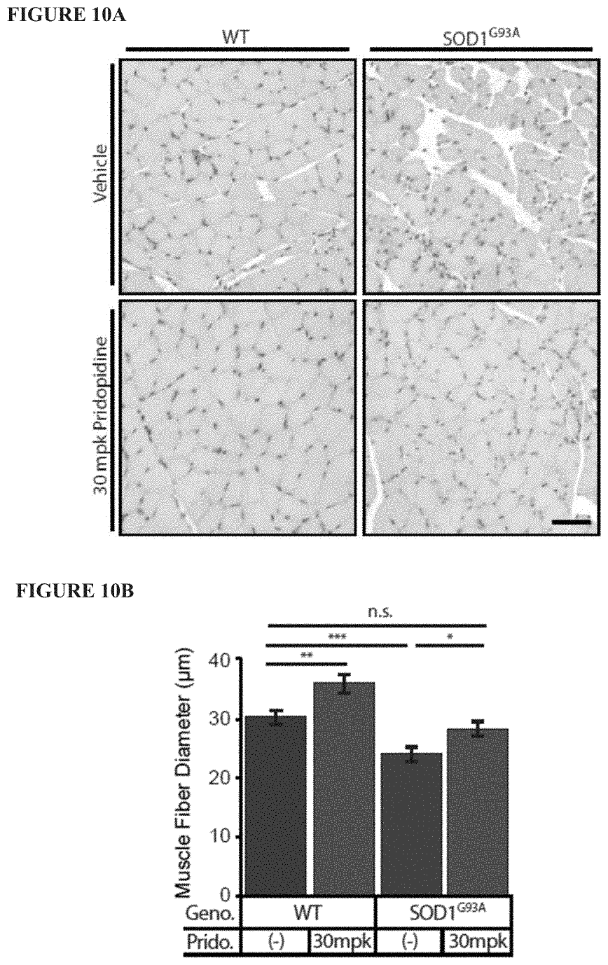

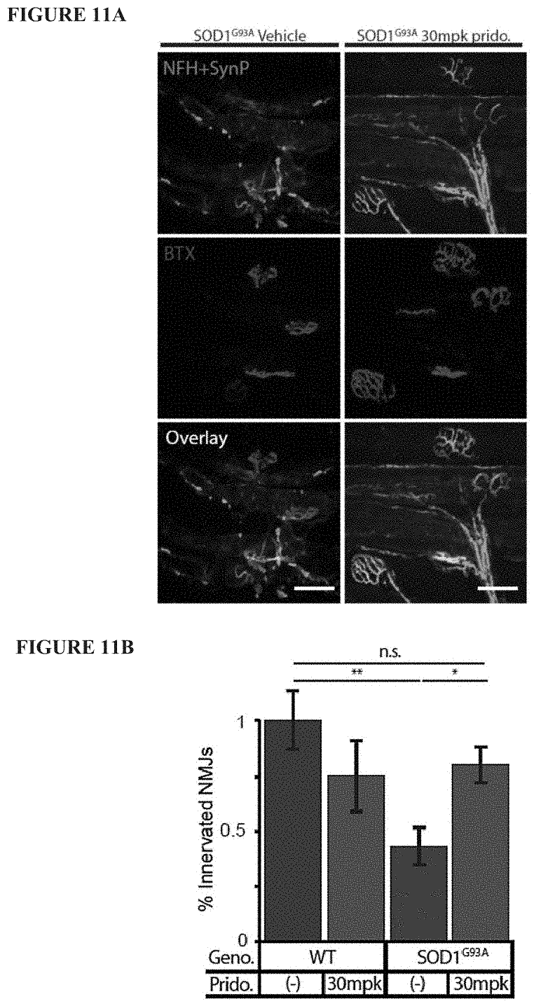

NMJ disruption and the subsequent skeletal muscle wasting are two main pathologies of ALS. Effect of pridopidine on muscle fiber atrophy and preservation of NMJs was evaluated in-vivo. Pre-symptomatic SOD1G93A mice and WI controls (5 weeks old) were treated with either saline, or pridopidine 30 mg/kg, by daily s.c administration for 11 weeks. The Gastrocnemius muscles from vehicle or pridopidine-treated (30 mg/kg s.c.) mice were extracted from the SOD1G93A and WT mice at age 16 weeks. Muscle cross-sections were stained with Hematoxylin & Eosin (H&E), and the mean muscle fiber diameter was quantified for each group (FIG. 10A). NMJ preservation was evaluated by confocal imaging of co-localizing pre (NFH+Synapsin-I (green)- and post-synaptic (AchR (BTX; red) markers and counting the number of fully innervated NMJs in gastrocnemius muscles (FIG. 11A).

### Results:

FIG. 10A: Representative images of H&E-stained cross-sections from Gastrocnemius muscle of mice from 4 groups: WI-vehicle treated, WT-30 mg/kg pridopidine treated, SOD1G93A-vehicle treated, and SOD1G93A-30 mg/kg pridopidine treated mice. Muscle histology of SOD1G93A-vehicle mice was poor and revealed a smaller diameter of muscle fiber as compared with WI-vehicle and WT-30 mg/kg pridopidine (FIGS. 10A-10B). Pridopidine (30 mg/kg, s.c daily administration) led to a significant ˜4 μm increase in the muscle fiber diameter in SOD1G93A and ˜5 μm in WI muscles.

NMJ preservation in muscles of SOD1G93A vehicle-treated mice indicates the expected massive ˜60% loss of NMJ and morphological changes in the post-synaptic apparatus in the muscles of SOD1G93A mice compared to WT mice (FIGS. 11A-11B). Strikingly, pridopidine treatment limited the loss of NMJs in SOD1G93A mice to 20%. Data are shown as mean ±SEM, *p-value <0.05; **p-value <0.01; ***p-value <0.001 (double-blind Student's t test).

Overall, these results demonstrate that pridopidine exerted neuroprotective effects in ALS cellular and animal models. In-vitro, in SOD1G93A MNs, pridopidine enhanced BDNF axonal transport, upregulated ERK activation, enhanced axonal growth, restored muscle innervation and improved NMJ formation and function. These neuroprotective effects were mediated by the S1R. In-vivo pridopidine treatment of SOD1G93A ALS mice reduced mutant SOD1 aggregation in spinal cord (a hallmark of the disease), increased the ALS-reduced muscle fiber diameter and preserved the degenerated NMJs observed in diseased tissue. These data support the use of pridopidine as a neuroprotective agent, and the S1R as a therapeutic target for the treatment of ALS patients. In the figures, abbreviations are as follows: Geno.=genotype (i.e. wild type (WT), mutant SOD1), Prido=pridopidine, mpk=milligram per kilogram.

### Experiment 7

### Treatment of ALS in a Human Subject

Periodically orally administering of pridopidine provides a clinically meaningful advantage in is reducing the symptoms of ALS in human subjects afflicted with ALS. Pridopidine therapy provides efficacy in treating the patient and is effective in at least one of the following embodiments
1. The therapy is effective in improving symptoms of ALS;
2. The therapy is effective in enhancing BDNF axonal transport in motor neurons and/or enhancing ERK activation;
3. The therapy is effective in improving NMJ formation and preservation, preserving NMJ structure, preserving NMJ function and/or improving innervation rate of muscle tissue;
4. The therapy is effective in enhancing motor neuron axonal growth and/or reducing axonal degeneration, including motor neuron axonal degeneration;
5. The therapy is effective in enhancing muscle cell survival, enhancing muscle fiber diameter and function, reduce progression of muscle fiber wasting, and/or improve muscle contraction; and or
6. The therapy is effective in reducing SOD1 aggregation and/or lessening pseudobulbar disease progression.

In some patients, the attending physician administers pridopidine and a second compound, wherein the second compound is second compound is riluzole, edaravone, dextromethorphan/quinidine. In some embodiments, the second compound is laquinimod.

### Experiment 8

### ALS Clinical Trial

The ALS Platform Trial is managed by the Healey Center for ALS at the Massachusetts General Hospital. This is a multicenter, multi-regimen, randomized, placebo-controlled, adaptive platform clinical trial evaluating the safety and efficacy of multiple investigational products simultaneously or sequentially in ALS.

Treatment duration of placebo-controlled regimens is a maximum of 24-weeks for each regimen. An optional open label extension (OLE) may be offered

The specific pridopidine regimen is based on cumulative preclinical and clinical studies that suggest a beneficial effect for pridopidine in ALS. Pridopidine acts primarily as a Sigma-1 Receptor (S1R) agonist. Pridopidine demonstrates neuroprotective properties in nonclinical models of neurodegenerative diseases, mediated by S1R.

The purpose of this clinical study of pridopidine is to evaluate the effect of pridopidine 45 mg BID on ALS disease progression including functional decline, bulbar function, muscle strength, function of upper and lower limb, voice characteristics, respiratory function and biomarker levels in participants with ALS.

The number of planned participants for the pridopidine regimen is 160.

There are 2 treatment groups for this regimen, active and placebo. Participants will be randomized in a 3:1 ratio to active treatment or placebo (i.e., 120 active: 40 placebo).

The maximum duration of the placebo-controlled treatment period is 24 weeks.

### Dosing Regimen

The proposed dose to be tested in the planned study is 45 mg twice daily (BID), taken in the morning and in the early afternoon (approximately 7 to 10 hours after the morning dose). There is a titration period leading up to the proposed dose whereby participants are initiating pridopidine at 45 mg QD and then increasing to 45 mg BID after 2 weeks.

### Study Objectives

### Primary Efficacy Objective:

To evaluate the efficacy of pridopidine as compared to placebo on ALS disease progression.

### Secondary Efficacy Objective:

To evaluate the effect of pridopidine on selected secondary measures of disease progression, including survival.

### Safety Objective:

To evaluate the safety of pridopidine in ALS patients.

### Exploratory Efficacy Objective:

To evaluate the effect of pridopidine on selected biomarkers and endpoints.

### Study Endpoints:

### Primary Efficacy Endpoint:

Change in disease severity as measured by the ALS Functional Rating Scale-Revised (ALSFRS-R) using a Bayesian repeated measures model that accounts for loss to follow-up due to mortality. Justification

The ALSFRS-R measures function in daily activities and is an established scale for monitoring disease progression in ALS. Each type of function is scored from 4 (normal) to 0 (no ability), with a maximum total score of 48 and a minimum total score of 0. Patients with higher scores have more physical function.

### Secondary Efficacy Endpoints:

1. Bulbar Function in Participants with Bulbar Dysfunction
   Rate of change in ALSFRS-R bulbar subdomain (Q1-Q3) score among participants with bulbar dysfunction at baseline, Each question is scored from 4 (normal) to 0 (no ability), with a maximum total score of 12 and a minimum total score of 0 for the bulbar subdomain. Patients with higher scores have more bulbar function.
2. Bulbar function in all Randomized participants.
   Rate of change in ALSFRS-R bulbar subdomain (Q1-Q3) score among all randomized participants. Each question is scored from 4 (normal) to 0 (no ability), with a maximum total score of 12 and a minimum total score of 0 for the bulbar subdomain. Patients with higher scores have more bulbar function.
3. Speech
4. Rate of change in the speech sub-score of the ALSFRS-R (Q1) among all randomized participants. The speech question is scored from 4 (normal) to 0 (no ability), with a maximum total score of 4 and a minimum total score of 0. Patients with higher scores have better speech. Respiratory function
   Rate of change in SVC maximum percent of predicted among all randomized participants,
5. Bulbar Function in participants with rapid pre-baseline progression
   Rate of change in ALSFRS-R bulbar subdomain (Q1-Q3) score among participants with pre-baseline slope ≥0.75 points/month, Each question is scored from 4 (normal) to 0 (no ability), with a maximum total score of 12 and a minimum total score of 0 for the bulbar subdomain. Patients with higher scores have more bulbar function.
6. Time to Bulbar Dysfunction
   Time from baseline to the first observed bulbar dysfunction as measured by an ALS Functional Rating Scale-Revised (ALSFRS-R) bulbar subdomain score of less than 12.
7. Muscle Strength
   Rate of change in muscle strength as measured isometrically using hand-held dynamometry HHD. Percent change from baseline among all randomized participants,
8. Survival
   Time to death or death equivalent

### Rationale

Decline in respiratory function is a direct result of the known pathophysiology of the ALS and demonstration of a treatment benefit on respiratory endpoints may also provide evidence of effectiveness.
Loss of strength is a hallmark of disease progression in ATS and meaningful differences in muscle strength should be supportive of an effect on measures of function in activities of daily living.

### Exploratory Endpoints:

Changes in quantitative voice characteristics,
Changes in biofluid biomarkers of neurodegeneration.
Changes in patient reported outcomes.

### Rationale

These endpoints provide greater understanding of ALS and may provide identification of surrogate endpoints that are reasonably likely to predict clinical benefit

### REFERENCES

Al-Saif et al. (2011) A Mutation in Sigma-1 Receptor Causes Juvenile Amyotrophic Lateral Sclerosis, ANN NEUROL; 70:913-919.

Banci et al. (2008) SOD1 and Amyotrophic Lateral Sclerosis: Mutations and Oligomerization, PLoS One 3(2):e1677.

Bernard-Marissal er al. (2015) Dysfunction in endoplasmic reticulummitochondria crosstalk underlies SIGMAR1 loss of function mediated motor neuron degeneration, BRAIN: 138; 875-890.

Bilsland et al. (2010) Deficits in axonal transport precede ALS symptoms in vivo, Proc Natl Acad Sci U S A. 107(47):20523-8.

Bonni A, et al. (1999) Cell survival promoted by the Ras-MAPK signaling pathway by transcription-dependent and -independent mechanisms, Science 286:1358-1362

Bozzoni et al. (2016) Amyotrophic lateral sclerosis and environmental factors”, Funct. Neurol. 31(1):7-19.

Brod et al. (2000) Combination therapy with glatiramer acetate (copolymer-1) and a type I interferon (IFN-α) does not improve experimental autoimmune encephalomyelitis. Annals of Neurology, 47:127-131.

Cedarbaum (1999) The ALSFRS-R.: a revised ALS functional rating scale that incorporates assessments of respiratory function. J. Neurol. Sci. 169(1-2):13-21.

De Vos et al. (2007) Familial amyotrophic lateral sclerosis-linked SOD1 mutants perturb fast axonal transport to reduce axonal mitochondria content. Hum Mol Genet. 16(242720-2728.

Eddings er al. (2019) Pridopidine protects neurons from mutant-huntingtin toxicity via the sigma-1 receptor. Neurobiol Dis. September; 129: 118-129.

Eykens C, Robberecht W (2015) The genetic basis of amyotrophic lateral sclerosis: recent breakthroughs, Adv Genomics Genetics 5:327-345.

Geva et al. (2016). Pridopidine activates neuroprotective pathways impaired in Huntington Disease. HMG 25(18): 3975-87.

Guidance for industry. in vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin. Pharm., Nov. 1999 <http://www.fda.gov/cber/gdins/inetabol.pdf>.

Hammarström P, et al, (2010) A fluorescent pentameric thiophene derivative detects in vitro-formed prefibrillar protein aggregates. Biochemistry 49:6838-45

Ionescu et al (2016) Compartmental microfluidic system for studying muscle-neuron communication and neuromuscular junction maintenance. (2016) European Journal of Cell Biology 95(2) 69-88.

Ionescu et al. (2019) Targeting the Sigma-1 Receptor via PridopidineAmelioratesCentralFeaturesof ALS Pathology in a SOD1^{G93A} Model, lonescu et al. Cell Death and Disease 10:210.

Izumi et al. (2018) Compound heterozygote mutations in the SIGMAR1 gene in an oldest-old patient with amyotrophic lateral sclerosis. Geriatr. Gerontol. Int. 18:1519-1520.

Langa F et al. (2003) Generation and phenotypic analysis of sigma receptor type I (sigma 1) knockout mice. Eur J Neurosci. 18:2188-96.

Maier et al. (2013) Differentiated NSC-34 motorneuron-like cells as experimental model for cholinergic neurodegeneration, Neurochem. Int. 62(8):1029-38.

Martel et al. (2016) From animal models to human disease: a genetic approach for personalized medicine in ALS, Acta Neuropathol. Commun. 4(1):70.

Mavlyutov et al. (2013) Lack of sigma-1 receptor exacerbates ALS progression in mice. Neuroscience June 14; 240: 129-134. McGoldrick et al. (2013) Rodent models of amyotrophic lateral sclerosis. BBA Basis of Disease 1832 (9):1421-1436.

Millecamps and Julien (2013) Axonal transport deficits and neurodegenerative diseases, Nat Rev Neurosci. 14:161-76.

Perlson, et al. (2009) A Switch in Retrograde Signaling from Survival to Stress in Rapid Onset Neurodegeneration. J Neurosci. 2009 29(31): 9903-9917.

Peters et al. (2015) Emerging mechanisms of molecular pathology in ALS, J. Clin. Invest. 125(5): 1767-1779.

Ponten, et al. (2010). In vivo pharmacology of the dopaminergic stabilizer pridopidine. Eur J Pharmacol. 644(1-3): 88-95.

Prause et al. (2013) Altered localization, abnormal modification and loss of function of Sigma receptor-1 in amyotrophic lateral sclerosis. Human Molecular Genetics, Vol 22, No. 8.

Riva et al. (2016) Recent advances in amyotrophic lateral sclerosis, J. Neurol. 263:1241-1254.

Ryskamp, et al (2017) The sigma-1 receptor mediates the beneficial effects of pridopidine in a mouse model of Huntington disease. Neurobiol of Disease 97(Pt A):46-59.

Ryskamp, et al (2018) PridopidinestabilizesmushroomspinesinmousemodelsofAlzheimer's diseasebyactingonthesigma-1receptor. Neurobiology of Disease 124: 489-504.

Sahlholm et al. (2013) The dopamine stabilizers ACR16 and (−)-OSU6162 display nanomolar affinities at the s-1 receptor. Molec Psychiatry 18, 12-14.

Sahlhohn et al. (2015) Pridopidine selectively occupies sigma-1 rather than dopamine D2 receptors at behaviorally active doses. Psychopharm. 232(18):3342-53.

Smith, et al. (2017). Enhanced Bulbar Function in Amyotrophic Lateral Sclerosis: The Nuedexta Treatment Trial. Neurotherapeutics, 14(3), 762-772.

Stegmann, G., et al (2020) Early detection and tracking of Bulbar changes in ALS via frequent and remote speech analysis, npj Digit. Med. 3, 132. Song et al. (2013) An update on amine oxidase inhibitors: Multifaceted drugs, Prog. Neuropyschopharmacol. Biol. Psychiatry 44:118-124.

Watanabe et al. (2016) Mitochondria-associated membrane collapse is a common pathomechanism in SIGMAR1- and SOD1-linked ALS. EMBO Molecular Medicine Vol 8, No 12: 1421-1437.

Zou et al. (2016) Toward precision medicine in amyotrophic lateral sclerosis, Ann. Transl. Med. 4(2):27.

Zahavi, et al. (2015) A compartmentalized microfluidic neuromuscular co-culture system reveals spatial aspects of GDNF functions. J. Cell Sci. 128, 1241-1252.

Riluzole—Drug Summary, PDR (Prescribers' Digital Reference), www.pdr.net/drug-summary/Rilutek-riluzole-526 accessed Jul. 28, 2017

Edaravone—Drug Summary, PDR (Prescribers' Digital Reference), www.pdr.net/drug-summary/Radicava-edaravone-24080 accessed Jul. 28, 2017

Dextromethorphan hydrobromide/quinidine sulfate—Drug Summary, PDR (Prescribers' Digital Reference), http://www.pdr.net/drug-summary/Nuedexta-dextromethorphan-hydrobromide-quinidine-sulfate-1344.3281 accessed Aug. 14, 2017

U.S. Pat. No. 7,923,459

U.S. Pat. No. RE46117

PCT Application Publication No. WO 2016/138135

PCT Application Publication No. WO 2017/015609

What is claimed is:

1. A method for maintaining, improving, or lessening the decline of ALS patient's functionality, respiratory function, muscle strength, bulbar function, speech or any combination thereof in a subject afflicted with amyotrophic lateral sclerosis (ALS), comprising administering to the subject a therapeutically acceptable amount of pridopidine.

2. The method of claim 1, wherein the ALS is sporadic ALS.

3. The method of claim 1, wherein ALS patient's functionality comprises speech, salivation, swallowing, handwriting, cutting food and handling utensils, dressing and hygiene, turning in bed and adjusting bed clothes, walking, climbing stairs, dyspnea, orthopnea, respiratory insufficiency or any combination thereof.

4. The method of claim 1, wherein said change in respiratory function is assessed by slow vital capacity (SVC).

5. The method of claim 1, wherein said maintaining, improving, or lessening the decline in muscle strength is measured isometrically using hand-held dynamometry (HHD) and/or bilateral Hand Grip.

6. The method of claim 1, wherein said maintaining, improving, or lessening the decline in bulbar function is measured by the ALSFRS-R bulbar subdomain (Q1-Q3) score.

7. The method of claim 1, wherein said maintaining, improving, or lessening the decline in bulbar function is measured by the CNS-BFS.

8. The method of claim 1, wherein said subject has bulbar disfunction.

9. the method of claim 1, wherein said subject has rapid pre-baseline progression.

10. The method of claim 1, wherein the amount of pridopidine if effective to change time to first evidence of bulbar dysfunction.

11. The method of claim 1, wherein the maintaining, improving, or lessening the decline in speech is measured by the ALSFRS-R speech domain score (Q1) or by automated algorithmic assessment of speech collected digitally.

12. The method of claim 1, wherein the maintaining, improving, or lessening the decline is measured by the ALS Functional Rating Scale-Revised (ALSFRS-R).

13. The method of claim 1, wherein the amount of pridopidine is administered daily, twice a week, three times a week or more often than once daily.

14. The method of-claim 1, wherein the amount of pridopidine is administered twice daily.

15. The method of claim 1, wherein the amount of pridopidine is administered orally.

16. The method of claim 1, wherein the amount of pridopidine administered is 10 mg per day to 90 mg per day.

17. The method of claim 1, wherein the pridopidine is pridopidine hydrochloride.

18. The method of claim 1, wherein the subject is a human subject.

19. The method of claim 1, further comprising administering to the subject a therapeutically effective amount of a second compound, wherein the second compound is riluzole, edaravone, dextromethorphan/quinidine, sodium phenylbutyrate (PB), tauroursodeoxycholic acid, sodium phenylbutyrate (PB)/tauroursodeoxycholic acid (i.e. AMX0035), Zilucoplan, Verdiperstat, CNM-Au8 nanocrystalline gold or IC14.

20. The method of claim 19, wherein the administration of the second compound precedes the administration of pridopidine.

21. The method of claim 19, wherein the administration of pridopidine precedes the administration of the second compound.

22. The method of claim 19, wherein the pridopidine is administered adjunctively to the second compound.

23. The method of claim 19, wherein the second compound is administered adjunctively to the pridopidine.
